SEQUENCE LISTING (14 sequences)

SEQ ID NO: 1 - RNA, 83 nt, homo sapiens
ccuuggaguaaaguagcagcacauaaugguuuguggauuuugaaaaggugcaggccauauugugcugccucaaaaauacaagg

SEQ ID NO: 2 - RNA, 22 nt, homo sapiens
uagcagcacauaaugguuugug

SEQ ID NO: 3 - RNA, 98 nt, homo sapiens
uugaggccuuaaaguacuguagcagcacaucaugguuuacaugcuacagucaagaugcgaaucauuauuugcugcucuagaaauuuaaggaaauucau

SEQ ID NO: 4 - RNA, 22 nt, homo sapiens
cgaaucauuauuugcugcucua

SEQ ID NO: 5 - RNA, 89 nt, homo sapiens
gucagcagugccuuagcagcacguaaauauuggcguuaagauucuaaaauuaucuccaguauuaacugugcugcugaaguaagguugac

SEQ ID NO: 6 - RNA, 22 nt, homo sapiens
uagcagcacguaaauauuggcg

SEQ ID NO: 7 - DNA, 24 bp, Artificial Sequence
  15aMFWD
ttttggggtattttatgttttagt

SEQ ID NO: 8 - DNA, 21 bp, Artificial Sequence
  15aMREV
ccgataataaccgtcatctcg

SEQ ID NO: 9 - DNA, 25 bp, Artificial Sequence
  15aUFWD
tttggggtattttatgttttagtgt

SEQ ID NO: 10 - DNA, 25 bp, Artificial Sequence
  15aUREV
acccaataataaccatcatctcata

SEQ ID NO: 11 - DNA, 22 bp, Artificial Sequence
  15bMFWD
aaggattcggagtcgaaatatc

SEQ ID NO: 12 - DNA, 25 bp, Artificial Sequence
  15bMREV
gaacaaaacaaaaaataaaaacgta

SEQ ID NO: 13 - DNA, 25 bp, Artificial Sequence
  15bUFWD
gtgaaggatttggagttgaaatatt

SEQ ID NO: 14 - DNA, 26 bp, Artificial Sequence
  15bUREV
caaacaaaacaaaaaataaaaacata

(19) United States
(12) Patent Application Publication
(10) Pub. No.: US 2023/0183808 A1
(43) Pub. Date: Jun. 15, 2023

(54) Methods of Detecting and Treating Cancers Characterized by Loss of miR15 and miR16 Expression

(71) Applicant: Carlo M. Croce, Columbus, OH (US)

(72) Inventor: Carlo M. Croce, Columbus, OH (US)

(21) Appl. No.: 17/919,904

(22) PCT Filed: Apr. 21, 2021
(86) PCT No.: PCT/US2021/028375

§ 371 Date: Oct. 19, 2022

Related U.S. Application Data
(60) Provisional application No. 63/013,254, filed on Apr. 21, 2020.

(51) Int. Cl. C12Q 1/6886

(52) U.S. Cl. CPC C12Q 1/6886; C12Q 2600/118; C12Q 2600/178

(57) ABSTRACT

Provided herein are, in various embodiments, methods of predicting a likelihood of a subject developing an acute myeloid leukemia (AML) or blast crisis (BC), methods of detecting a subject having AML or BC cells susceptible to treatment with a B cell-specific Moloney murine leukemia virus integration site 1 (Bmi-1) inhibitor, a B-cell lymphoma 2 (Bel -2) inhibitor and/or a Receptor Tyrosine Kinase Like Orphan Receptor 1 (RORI) inhibitor, methods of stratifying subjects having Myelodysplastic syndrome (MDS) or chronic phase chronic myeloid leukemia (CML), and methods of preparing a sample that is useful for performing the disclosed methods. The present invention also provides methods of treating a subject having a leukemia (e.g., MDS AML or CML) with a therapy that increases the expression or activity of miR-15a, miR-15b, miR-16-1 and/or miR-16-2 gene product, reduces the expression or activity of a target of said gene product, or a combination thereof.

### RELATED APPLICATION(S)

This application claims the benefit of U.S. Provisional Application No. 63/013,254, filed on Apr. 21, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

a) File name: 5050_1002_001_Sequence_Listing.txt; created Apr. 15, 2021, 3 KB in size.

### GOVERNMENT SUPPORT

This invention was made with government support under Grant Nos. P30CA16058 and R35CA197706 awarded by the National Cancer Institute. The government has certain rights in the invention.

### BACKGROUND

Myelodysplastic Syndrome (MDS) is a class of clonal hematopoietic stem cell disorders characterized by cytopenia, myelodysplasia and ineffective hematopoiesis. MDSs are associated with increased risk of progression to acute myeloid leukemia (AML). AML is a biologically and clinically heterogeneous disease and the most common acute leukemia in adults. The pathogenesis of AML involves the abnormal proliferation and differentiation of a clonal population of myeloid stem cells (De Kouchkovsky I. and Abdul-Hay, M., Blood Cancer Journal 6, e441 (2016)). Prognosis in AML patients, particularly patients 65 years or older, remains poor.

Chronic myelogenous leukemia (CML) is a malignant disorder characterized by an increase in myeloid cells that maintains their ability to differentiate (Faderl et al., N Engl JMed. 341(3): 164-72 (1999)). It represents almost 20% of all leukemias in adults and with appropriate targeted therapy has an indolent course (Kantarjian et al., Am JMed. 83(3):445-54 (1987)). CML proceeds in three phases: chronic, accelerated, and blastic (blast crisis, BC) (Sawyers, N Engl J Med.340(17):1330-40 (1999)). Untreated CMLs progress to an accelerated phase and then to a BC, which is essentially incurable (Radich, Hematology Am. Soc. Hematol. Educ. Program, 384-91 (2007)). At present, mechanisms responsible for the progression of chronic phase CML to BC are not fully understood.

### SUMMARY

There is a critical need to improve the understanding of the biology of leukemia, for example, Myelodysplastic syndrome (MDS), acute myeloid leukemia (AML) and chronic myeloid leukemia (CML), and to develop improved therapies for treatment of these disorders.

The invention disclosed herein is based, at least in part, on the discovery that reduced expression of miR-15a, miR-15b, miR-16-1, miR-16-2, or a combination thereof is associated with transformation of MDS to AML and transformation of chronic phase CML to CML blast crisis (BC). Accordingly, the present invention generally relates to methods of detecting and treating cancer (e.g., a leukemia such as MDS, AML, chronic phase CML or BC) that are characterized by a reduction in (e.g., a loss of) expression of miR-15a, miR-15b, miR-16-1, miR-16-2, or a combination thereof.

One aspect of the invention relates to a method of identifying a MDS that is likely to transform into an AML in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a MDS that is likely to transform into an AML.

Another aspect of the invention relates to a method of identifying a subject having a MDS or an AML as a candidate for a treatment comprising a B-cell lymphoma 2 (Bcl-2) inhibitor, a Receptor Tyrosine Kinase Like Orphan Receptor 1 (ROR1) inhibitor, or both, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bcl-2 inhibitor, a ROR1 inhibitor, or both.

Another aspect of the invention relates to a method of stratifying a set of subjects having MDS for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in samples from a subset of subjects, identifies the subset of subjects who are candidates for a treatment comprising a Bcl-2 inhibitor, a ROR1 inhibitor, or both.

Another aspect of the invention relates to a method of treating a subject having an AML, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

Another aspect of the invention relates to a method of treating a subject having a MDS, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

Another aspect of the invention relates to a method of preparing a sample that is useful for predicting a likelihood of a subject of developing an AML, comprising:
a) obtaining or having obtained the sample from the subject; and
b) reverse transcribing a miRNA from the sample to provide the target oligodeoxynucleotides, wherein the miRNA is miR-15a, miR-15b, miR-16-1, miR-16-2 or a combination thereof.

Another aspect of the invention relates to a method of preparing a sample that is useful for detecting a subject having AML cells susceptible to treatment with a Bcl-2 inhibitor, a ROR1 inhibitor, or both, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

Another aspect of the invention relates to a method of preparing samples that are useful for stratifying a set of subjects having MDS for treatment, comprising:
a) obtaining or having obtained the samples from the subjects; and
b) reverse transcribing a miRNA from the individual samples to provide target oligodeoxynucleotides, wherein the miRNA is miR-15a, miR-15b, miR-16-1, miR-16-2 or a combination thereof.

Another aspect of the invention relates to a method of identifying a chronic phase CML that is likely to transform into a BC in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a chronic phase CML that is likely to transform into a BC.

Another aspect of the invention relates to a method of identifying a subject having a chronic phase CML or a BC as a candidate for a treatment comprising a Bmi-1 inhibitor, a Bcl-2 inhibitor, a ROR1 inhibitor, or a combination thereof, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bmi-1 inhibitor, a Bcl-2 inhibitor, a ROR1 inhibitor, or a combination thereof.

Another aspect of the invention relates to a method of stratifying a set of subjects having chronic phase CML for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in samples from a subset of subjects, identifies the subset of subjects who are candidates for a treatment comprising a Bmi-1 inhibitor, a Bcl-2 inhibitor, a ROR1 inhibitor, or a combination thereof.

Another aspect of the invention relates to a method of treating a subject having a BC, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

Another aspect of the invention relates to a method of treating a subject having a chronic phase CML, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

Another aspect of the invention relates to a method of preparing a sample that is useful for predicting a likelihood of a subject of developing a BC, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

Another aspect of the invention relates to a method of preparing a sample that is useful for detecting a subject having BC cells susceptible to treatment with a Bmi-1 inhibitor, a Bcl-2 inhibitor, a ROR1 inhibitor, or a combination thereof, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

Another aspect of the invention relates to a method of preparing samples that are useful for stratifying a set of subjects having chronic phase CML for treatment, comprising:
a) obtaining or having obtained the samples from the subjects; and
b) reverse transcribing a miRNA from the individual samples to provide target oligodeoxynucleotides, wherein the miRNA is miR-15a, miR-15b, miR-16-1, miR-16-2 or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show miR-15/16 cluster expression in MDS, MDS_T, MDS-AML and AML patients. Box plots of miR-15a (statistical analysis for MDS, MDS-T and MDS-AML Kruskal Adj. p = 7.25e-06; statistical analysis for all groups Kruskal Adj. p = 1.02e-09) (FIG. 1A), miR-15b (statistical analysis for MDS, MDS-T and MDS-AML Kruskal Adj. p = 7.4e-07; statistical analysis for all groups Kruskal Adj. P-value = 1.02e-09) (FIG. 1B), and miR-16 (statistical analysis for MDS, MDS-T and MDS-AML Kruskal Adj. p = 1.81e-05; statistical analysis for all groups Kruskal Adj. p = 1.47e-06) (FIG. 1C) expression by qRT-PCR. Kruskal-Wallis rank sum test was employed for statistical significance. FIGS. 1D-1F show a conditional inference tree (CTree) of the diagnostic classifier between MDS and MDS-T based on miR-15a (p = 0.014) (FIG. 1D), miR-15b (p < 0.001) (FIG. 1E), and miR-16 (p = 0.001) (FIG. 1F) expression. FIG. 1G is a histogram representing the percentage of AML patients with miR-15a and/or miR-15b expression lower (blue, green and orange bars) or greater (red bar) than the miR-15a and miR-15b expression median (50^{th} percentile) among AML patients. FIG. 1H is a histogram representing, among AML patients with miR-15a OR miR-15b expression lower than their median (orange bar from FIG. 1G), the percentage of AML patients with miR-15a or miR-15b expression lower than the first quartile (25^{th} percentile) or lower than median (50^{th} percentile). Abbreviation: perc, percentile. FIG. 1I shows Kaplan Meier overall survival (OS) analysis of AML patients based on the status of miR-15a, miR-15b and miR-16 expression (miR-15a, miR-15b and miR-16 expression greater than the median (n = 18) in red and miR-15a, miR-15b and miR-16 expression lower than the median (n = 19) in blue) (p = 0.046). For each subgroup, the differences between the curves were determined by two-sided log-rank tests. The tick marks indicate the censored subjects.

FIGS. 2A-2J show miR-15a, miR-15b and miR-16 expression in AML patients. FIG. 2A shows that the samples are classified in the terminal nodes of the decision tree: MDS (yellow) or MDS-T (green) based on their miR-16 and miR-15a expression. FIGS. 2B-2D are box plots of miR-15a (p = 0.04) (FIG. 2B), miR-15b (p = 1.98e-05) (FIG. 2C) and miR-16 (p = 0.0007) (FIG. 2D) expression by qRT-PCR in AML patients with NPM1 wild type (NEG, blue) versus patients with NPM1 mutation (POS, red). Wilcoxon rank-sum test was employed for statistical significance. FIGS. 2E-2G are box plots of miR-15a (p = 6.6e-05) (FIG. 2E), miR-15b (p = 0.00046) (FIG. 2F) and miR-16 (p = 7.28e-07) (FIG. 2G) expression in AML patients from TCGA database with NPM1 wild type (NEG, blue) versus patients with NPM1 mutation (POS, red). Wilcoxon rank-sum test was employed for statistical significance. FIGS. 2H-2J are box plot of miR-15a (p = 0.56) (FIG. 2H), miR-15b (p = 0.06) (FIG. 21) and miR-16 (p = 0.36) (FIG. 2J) expression among the cytogenetics abnormalities of AML patients. Kruskal-Wallis rank sum test was employed for each non-parametric multivariate analysis.

FIGS. 3A-3D show expression and validation of predicted target of miR-15/16 cluster in AML cell lines. FIG. 3A shows qRT-PCR analysis of miR-15a, miR-15b and miR-16 in AML cell lines compared to CD34+ cells from healthy donors. FIG. 3B shows immunoblotting of ROR-1 and Bcl-2 performed on AML cell lines and on CD34+ cells lysates. β-actin was used as a normalizer. FIG. 3C shows relative quantification of ROR-1 and Bcl-2 expression respect to β-actin loading control. FIG. 3D shows representative flow cytometry analysis of AML cell lines stained by annexin-V-FITC and propidium iodide after ABT-199 (venetoclax) treatment for 48 hours.

FIGS. 4A-4D show validation of predicted targets of miR-15/16 cluster in AML cell lines. FIGS. 4A and 4C are immunoblotting of Mcl-1 (FIG. 4A) and mTOR, PI3K p85 and Cyclin D1 (FIG. 4C) performed on AML cell lines and on CD34+ cells lysates. β-actin was used as a normalizer. FIGS. 4B and 4D show relative quantification of Mcl-1 (FIG. 4B) and mTOR, PI3K p85 and Cyclin D1 (FIG. 4D) expression respect to β-actin loading control.

FIGS. 5A-5F show genetic and epigenomic screening of AML cell lines. FIG. 5A shows Southern blot analysis of miR-15a and miR-15b in AML cell lines compared to PB Normal (normal peripheral blood Lymphocytes). TBP was used as a loading control (TATA-binding protein). FIG. 5B shows relative quantification of miR-15a and miR-15b expression relative to TBP loading control from Southern blot assay. FIG. 5C shows bisulfite genomic sequencing analysis after methylation-specific PCR of miR-15a CpG island in AML cell lines. FIG. 5D shows expression of miR-15a by qRT-PCR in untreated (CNT) or treated with DNA demethylating agent 5-aza-2′deoxycytidine cell lines. FIG. 5E shows bisulfite genomic sequencing analysis after methylation-specific PCR of miR-15b CpG island in AML cell lines. FIG. 5F shows expression of miR-15b by qRT-PCR in untreated (CNT) or treated with DNA demethylating agent 5-aza-2′deoxycytidine cell lines. In FIGS. 5C and 5E, at least 3 clones are analyzed for each cell line. The CpG island is pictured and each vertical bar represent a single CpG (depicted by *) or a single non-CpG site. Black (CpG site) and grey (non-CpG site) squares represent methylated CpG and white squares represent unmethylated CpG.

FIGS. 6A-6B show quantitative real time PCR copy number assays in AML cell lines (FIG. 6A) and AML patients (FIG. 6B). Custom Taqman assays were used to examine copy number variations within the miR-15a and miR-15b genes in DNA samples.

FIGS. 7A-7D show validation of predicted target of miR-15/16 cluster in AML patients. FIG. 7A shows Western blot analysis of mTOR, PI3K p85 and Bcl-2 performed on AML patients and on PBMC and CD34+ cells lysates. β-actin was used as a normalizer. FIG. 7B shows relative quantification of mTOR, PI3K p85 and Bcl-2 expression respect to β-actin loading control. FIGS. 7C-7D are scatter plots of the Spearman’s correlation analyses for the CCND2 gene and miR-15a/b in AML samples. R indicates the Pearson’s product-moment correlation coefficient and p indicates the p-value. The curves indicate the linear regression lines of the scatter plots.

FIGS. 8A-8B are scatter plots of the Spearman’s correlation analyses for the CCND1 gene and miR-15a/b in AML samples. R indicates the Pearson’s product-moment correlation coefficient and p indicates the p-value. The curves indicate the linear regression lines of the scatter plots.

FIGS. 9A-9F show MiR-15/16 cluster expression in CML patients. MicroRNAs expression by qRT-PCR in CD34+ healthy controls and CML patients’ cells in CP (n = 17) and in BC (n = 22). Box plots of miR-15a (statistical analysis for all groups Kruskal-Wallis P value = 0.0005431) (FIG. 9A), miR-15b (statistical analysis for all groups Kruskal-Wallis P value = 8.292 × 10⁻⁵) (FIG. 9B), miR-16 (statistical analysis for all groups Kruskal-Wallis P value = 0.0003215) (FIG. 9C). FIGS. 9D-9F show MicroRNAs expression by qRT-PCR of paired CML patients’ cells (n = 11) in CP and BC. Bipartite graph of miR-15a (one-way Wilcoxon signed-rank P value = 0.021) (FIG. 9D), miR-15b (one-way Wilcoxon signed-rank P value = 0.02686) (FIG. 9E), miR-16 (one-way Wilcoxon signed-rank P value = 0.01611) (FIG. 9F). n.s., not significant. *, 0.01< P value ≤ 0.05; **, 0.001 < P value ≤ 0.01; ***, 0.0001 < P value ≤ 0.001; ****P value ≤ 0.0001.

FIGS. 10A-10E show MiR-15/16 targets expression in CML patients. (FIG. 10A) Immunoblotting of ROR1, Bmi-1, and Bcl-2 performed on five CML patients’ cells in CP and five CML patients’ cells in BC. Glyceraldehyde-3-phosphate dehydrogenase (GAPDH) was used as a normalizer. (FIG. 10B) Densitometry relative quantification of ROR1, Bmi-1, and Bcl-2 expression respect to GAPDH loading control. CML patients’ cells in CP and in BC were grouped. One-way Wilcoxon rank-sum test was applied. (FIGS. 10C and 10D) Gene expression by qRT-PCR in CML patients’ cells in CP (n = 17), in BC (n = 20) and CD34+ cells from healthy donors (n = 4). Box plots expression of BMI1 (statistical analysis for all groups Kruskal-Wallis P value = 0.00027) (FIG. 10C) and BCL2 (statistical analysis for all groups Kruskal-Wallis P value = 4.37 × 10⁻⁵) (FIG. 10D). (FIG. 10E) Western blot analysis of ROR1, Bmi-1, and Bcl-2 performed on three paired CML patients’ cells in CP, BC, and CD34+ from two healthy donors. β-actin was used as a normalizer. *, 0.01 < P value ≤ 0.05; **, 0.001 < P value ≤ 0.01; ***, 0.0001 < P value ≤ 0.001; ****P value ≤ 0.0001.

### DETAILED DESCRIPTION

A description of example embodiments follows.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

In one aspect, the present invention provides a method of identifying a cancer that is likely to transform in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein the cancer is not chronic lymphocytic leukemia (CLL), and wherein a reduction in the level of the miR gene product relative to a threshold level, an absence of a detectable level of the miR gene product, or an allelic loss of the miR gene, in the sample from the subject, indicates that the subject has a cancer that is likely to transform.

In another aspect, the present invention provides a method of identifying a cancer that is likely to transform in a subject, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying a cancer that is likely to transform in the subject when there is a reduction in the level of the miR gene product relative to a threshold level, an absence of a detectable level of the miR gene product, or an allelic loss of the miR gene, in the sample from the subject,
wherein the cancer is not chronic lymphocytic leukemia (CLL).

In some embodiments, the method identifies a Myelodysplastic syndrome (MDS) that is likely to transform into an acute myeloid leukemia (AML) in the subject. In some embodiments, the method identifies a chronic phase chronic myeloid leukemia (CML) that is likely to transform into a blast crisis (BC) in the subject.

In another aspect, the present invention provides a method of identifying a MDS that is likely to transform into an AML in a subject, comprising determining, in a sample from the subj ect:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a MDS that is likely to transform into an AML.

In another aspect, the present invention provides a method of identifying a MDS that is likely to transform into an AML in a subject, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b),
b) identifying a MDS that is likely to transform into an AML in the subject when there is a reduction in the level of the miR gene product relative to a threshold level, an absence of a detectable level of the miR gene product, or an allelic loss of the miR gene, in the sample from the subject.

In another aspect, the present invention provides a method of identifying a chronic phase CML that is likely to transform into a BC in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a chronic phase CML that is likely to transform into a BC.

In another aspect, the present invention provides a method of identifying a chronic phase CML that is likely to transform into a BC in a subject, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying a chronic phase CML that is likely to transform into a BC in the subject when there is a reduction in the level of the miR gene product relative to a threshold level, an absence of a detectable level of the miR gene product, or an allelic loss of the miR gene, in the sample from the subject.

The term “subject” refers to a mammal (e.g., human, dog, cat, horse, cow, mouse, rat). Preferably, the subject is a human (e.g., a human who has, or is at risk for developing, an AML or a BC). A “subject in need thereof” refers to a subject who has, or is at risk of cancer transformation (e.g., developing, an AML or a BC). A skilled medical professional (e.g., physician) can readily determine whether a subject has, or is at risk for developing, a cancer (e.g., a MDS, an AML, a chronic phase CML or a BC), for example, using the methods described herein. In some embodiments, the subject has a MDS. In some embodiments, the subject is suspected of having a MDS. In some embodiments, the subject has a chronic phase CML. In some embodiments, the subject is suspected of having a chronic phase CML. In some embodiments, the subject is a mammal. In some embodiments, the mammal is a human (e.g., a human patient).

In some embodiments, the subject has a loss of miR-15a/16-1 on chromosome 13q14, a loss of miR-15b/16-2 on chromosome 3q25, or both. In some embodiments, the subject has a loss of miR-15a/16-1 on chromosome 13q14. In some embodiments, the subject has a loss of miR-15b/16-2 on chromosome 3q25. In some embodiments, the subject has a loss of miR-15a/16-1 on chromosome 13q14 and a loss of miR-15b/16-2 on chromosome 3q25. In some embodiments, the loss of miR-15a/16-1 on chromosome 13q14, the loss of miR-15b/16-2 on chromosome 3q25, or both, in the subject, is single allelic. In other embodiments, the loss of miR-15a/16-1 on chromosome 13q14, the loss of miR-15b/16-2 on chromosome 3q25, or both, in the subject, is biallelic.

In some embodiments, the sample comprises a tissue sample. In some embodiments, the tissue sample comprises bone marrow cells (e.g., bone marrow aspiration and/or biopsy). A tissue sample can be removed from the subject (e.g., a MDS or chronic phase CML patient) by conventional biopsy techniques. In some embodiments, the tissue sample is prepared using microdissection. In some embodiments, the sample comprises a blood sample. In some embodiments, the sample (tissue or blood sample) is obtained from the subject prior to initiation of a therapeutic treatment (e.g., radiotherapy or chemotherapy).

In some embodiments, determining the status of the miR gene in the sample comprises detecting a loss of miR-15a/16-1 on chromosome 13q14, determining the status of the miR gene in the sample comprises detecting a loss of miR-15b/16-2 on chromosome 3q25, or both.

In some embodiments, determining the status of the miR gene in the sample comprises measuring the level of the miR gene product. The term “miR gene product” includes both the unprocessed, or precursor, RNA product and the processed, or mature, RNA product of a miR gene. In some embodiments, the miR gene product comprises precursor miRNA (e.g., unprocessed, precursor RNA product of a wild type human miR gene). In some embodiments, the miR gene product comprises mature miRNA (e.g., processed, mature RNA product of a wild type human miR gene). A miR16 gene product can be either a miR16-1 gene product or a miR16-2 gene product, or both.

The sequences of precursor wild type human miR-15a, miR-15b and miR-16 are set forth in SEQ ID NO: 1, SEQ ID NO: 3 and SEQ ID NO: 5, respectively (Table 4). The sequences of mature wild type human miR-15a, miR-15b and miR-16 are set forth in SEQ ID NO: 2, SEQ ID NO: 4 and SEQ ID NO: 6, respectively (Table 4). The miR-16-1 and miR-16-2 precursor and mature sequences are identical and are represented by the miR-16 precursor and mature sequences shown in Table 4.

The level of a miR gene product in the sample can be measured using any technique suitable for detecting RNA expression levels in a biological sample. In some embodiments, the level of the miR gene product is measured using an assay selected from the group consisting of northern blot analysis, in situ hybridization, a microarray assay (e.g., a miRNA assay, such as an nCounter® miRNA assay available from NanoString Technologies, Inc. (Seattle, WA), or a variation thereof) and quantitative reverse transcriptase polymerase chain reaction (RT-qPCR). In certain embodiments, the level of the miR gene product is measured using RT-qPCR.

In some embodiments, the threshold level is the level of the corresponding miR gene product in a control sample or a reference standard.

In some embodiments, the threshold level is the level of the corresponding miR gene product in a control sample. In some embodiments, the control sample (e.g., tissue or blood sample) comprises unaffected tissue from the subject. In some embodiments, the control sample comprises tissue or blood from an unaffected subject or a population of unaffected subjects. An unaffected subject is a healthy subject, a subject who is not diagnosed with the cancer (e.g., MDS or chronic phase CML) or a subject who does not have cancer (e.g., MDS or chronic phase CML). In some embodiments, the control sample (e.g., tissue or blood sample) is processed along with the sample from the subject. In other embodiments, the control sample is processed separately (e.g., at an earlier or a later time) from the test sample.

In some embodiments, the threshold level is the level of a reference standard. The term “reference standard” can be, for example, a mean, an average, a numerical mean or range of numerical means, a numerical pattern, a graphical pattern or the corresponding miRNA expression level derived from a reference subject (e.g., an unaffected subject) or reference population (e.g., a population of unaffected subjects).

In some embodiments, the miR gene product comprises miR-15a.

In some embodiments, the level of miR-15a in the sample is less than about 50% of the threshold miR-15a level, e.g., less than about: 45%, 40%, 35%, 30%, 25%, 20%, 15%, 10% or 5%, of the threshold miR-15a level, or about: 50%, 45%, 40%, 35%, 30%, 25%, 20%, 15%, 10% or 5%, of the threshold miR-15a level. In some embodiments, the level of miR-15a in the sample is about 5-50% of the threshold miR-15a level, e.g., about: 5-45%, 10-45%, 10-40%, 15-40%, 15-35%, 20-35% or 20-30% of the threshold miR-15a level. In some embodiments, the level of miR-15a in the sample is about: 50%, 55%, 60%, 65%, 70%, 75%, 80%, 85%, 90% or 95%, of the threshold miR-15a level.

In some embodiments, the miR gene product comprises miR-15b.

In some embodiments, the level of miR-15b in the sample is less than about 50% of the threshold miR-15b level, e.g., less than about: 45%, 40%, 35%, 30%, 25%, 20%, 15%, 10% or 5%, of the threshold miR-15b level, or about: 50%, 45%, 40%, 35%, 30%, 25%, 20%, 15%, 10% or 5%, of the threshold miR-15b level. In some embodiments, the level of miR-15b in the sample is about 5-50% of the threshold miR-15b level, e.g., about: 5-45%, 10-45%, 10-40%, 15-40%, 15-35%, 20-35% or 20-30% of the threshold miR-15a level. In some embodiments, the level of miR-15b in the sample is about: 50%, 55%, 60%, 65%, 70%, 75%, 80%, 85%, 90% or 95%, of the threshold miR-15b level.

In some embodiments, the miR gene product comprises miR-16.

In some embodiments, the level of miR-16 in the sample is less than about 50% of the threshold miR-16 level, e.g., less than about: 45%, 40%, 35%, 30%, 25%, 20%, 15%, 10% or 5%, of the threshold miR-16 level, or about: 50%, 45%, 40%, 35%, 30%, 25%, 20%, 15%, 10% or 5%, of the threshold miR-16 level. In some embodiments, the level of miR-16 in the sample is about 5-50% of the threshold miR-16 level, e.g., about: 5-45%, 10-45%, 10-40%, 15-40%, 15-35%, 20-35% or 20-30% of the threshold miR-16 level. In some embodiments, the level of miR-16 in the sample is about: 50%, 55%, 60%, 65%, 70%, 75%, 80%, 85%, 90% or 95%, of the threshold miR-16 level.

In some embodiments, the method comprises determining the levels of each of miR-15a, miR-15b and miR-16 gene products in the sample.

In some embodiments, the method further comprises administering to the subject:
a) one or more agents that increase the expression or activity of the miR gene product;
b) one or more agents that that reduce the expression or activity of a target of the miR gene product; or
c) a combination of a) and b),
when there is a reduction in the level of the miR gene product relative to the threshold level, an absence of a detectable level of the miR gene product, or an allelic loss of the miR gene, in the sample from the subject.

In some embodiments, the method further comprises administering an effective amount of the one or more agents that increase the expression or activity of miR-15a, miR-15b, miR-16-1, miR-16-2, or a combination thereof. In some embodiments, the method further comprises administering an effective amount of the one or more agents that increase the expression or activity of miR-15a, miR-15b, miR-16-1 and miR-16-2. For example, nucleic acids encoding one or more of miR-15a, miR-15b, miR-16-1 and miR-16-2 can be delivered to cells of a subject receiving treatment (e.g., by a gene therapy method), wherein the nucleic acids are expressed in the cells of the subject upon delivery. The nucleic acids can be delivered by a variety of gene delivery constructs known in the art, such as plasmids, vectors (e.g., viral vectors, such as AAV, and non-viral vectors), and naked DNA. The miR-15a, miR-15b, miR-16-1 and/or miR-16-2 can be delivered and/or expressed using one nucleic acid encoding the miR gene products, or using separate nucleic acids for each miR gene product. The nucleic acids can be encapsulated in a suitable delivery vehicle (e.g., a liposome, a nanoparticle). In some embodiments, the method comprises introducing one or more nucleic acids encoding miR-15a, miR-15b, miR-16-1, miR-16-2, or a combination thereof, into hematopoietic cells of the subject (e.g., using nanoparticles).

In some embodiments, the method further comprises administering an effective amount of the one or more agents that that reduce the expression or activity of a target of the miR gene product. The target can be a direct target of miR-15a, miR-15b, miR-16-1, miR-16-2, or a combination thereof, or an indirect target of miR-15a, miR-15b, miR-16-1, miR-16-2, or a combination thereof.

In some embodiments, the subject has a MDS, and the method further comprises administering an effective amount of one or more agents that reduce the expression or activity of a target selected from the group consisting of B-cell lymphoma 2 (Bcl-2), Receptor Tyrosine Kinase Like Orphan Receptor 1 (ROR1), Cyclin D1, myeloid cell leukemia 1 (Mcl-1) and combinations thereof. In some embodiments, the target is Bcl-2. In some embodiments, the target is ROR1.

In some embodiments, the subject has a chronic phase CML, and the method further comprises administering an effective amount of one or more agents that reduce the expression or activity of a target selected from the group consisting of B cell-specific Moloney murine leukemia virus integration site 1 (Bmi-1), Bcl-2, ROR1, Cyclin D1, Mcl-1 and combinations thereof. In some embodiments, the one or more agents are selected from the group consisting of Bmi-1, Bcl-2, ROR1 and combinations thereof.

In some embodiments, the method further comprises administering an effective amount of a Bmi-1 inhibitor, or a pharmaceutically acceptable salt thereof. In some embodiments, the Bmi-1 inhibitor is selected from the group consisting of artemisinin, PRT4165, PTC209, PTC596 and QW24.

In some embodiments, the method further comprises administering an effective amount of a Bcl-2 inhibitor, or a pharmaceutically acceptable salt thereof. In some embodiments, the Bcl-2 inhibitor is selected from the group consisting of 2,3-DCPE, 2-methoxy-antimycin A3, 3-bromopyruvic acid, A-1210477, AG 1024, AT-101, EM20-25, (-)-Epigallocatechin Gallate, Fluvastatin, FX1, gambogic acid, Gossypol, Marinopyrrole A (Maritoclax), navitoclax (ABT-263), Nilotinib or Nilotinib-d3, Piped ongumine, UMI-77, venetoclax, YC137 and combinations thereof. Additional non-limiting examples of Bcl-2 inhibitors include antisense oligonucleotide drugs (e.g., oblimersen), Bax activators (e.g., BAM7), BCL-xl/BH3 domain interaction inhibitors (e.g., BH3I-1), BCL-xl inhibitors (e.g., A-1331852 or A- 1155463), BH3 mimetics (e.g., ABT-737, ABT-737-d8, flavonoids (e.g., Licochalcone A), navitoclax/ABT-263 or ABT-263-d8), non-peptidic ligands of BCL-2 (e.g., HA14-1), non-peptide inhibitors (e.g., TW-37), pan-BCL-2 inhibitors (e.g., Sabutoclax, Obatoclax) and small molecule BCL-2/BH4 domain antagonists (e.g., BDA-366).

In some embodiments, the Bcl-2 inhibitor comprises venetoclax, or a pharmaceutically acceptable salt thereof. The Bcl-2 inhibitor venetoclax (ABT-199, Venclexta, Venclyxto) is used for treating hematological cancers, including AML and BC. A skilled physician or other medical/healthcare professional can readily determine an appropriate dose (e.g., therapeutically effective amount) of venetoclax to be administered to a subject.

In some embodiments, the method further comprises administering an effective amount of a ROR-1 inhibitor. In some embodiments, the ROR-1 inhibitor comprises a monoclonal antibody against ROR-1, or antigen binding fragment thereof. Additional non-limiting examples of ROR-1 inhibitors include KAN0439834 and Strictinin.

In some embodiments, the methods disclosed herein comprise the administration of two or more therapeutic agents that reduce the expression or activity of one or more targets of the miR gene product. In some embodiments, the method comprises administering an effective amount of a Bcl-2 inhibitor and an effective amount of a ROR1 inhibitor. In some embodiments, the method comprises administering an effective amount of venetoclax and an effective amount of a monoclonal antibody against ROR-1. In some embodiments, the method comprises administering an effective amount of a Bmi-1 inhibitor and an effective amount of a Bcl-2 inhibitor. In some embodiments, the method comprises administering an effective amount of a Bmi-1 inhibitor and an effective amount of a ROR1 inhibitor. In some embodiments, the method comprises administering an effective amount of a Bmi-1 inhibitor, an effective amount of a ROR1 inhibitor and an effective amount of a ROR1 inhibitor.

When administering a combination therapy, one agent can be administered before, after or concurrently with the other agent. When co-administered simultaneously (e.g., concurrently), the two or more agents can be in separate formulations or the same formulation. Alternatively, the two or more agents can be administered sequentially, as separate compositions, within an appropriate time frame as determined by a skilled clinician (e.g., a time sufficient to allow an overlap of the pharmaceutical effects of the therapies).

Two or more agents can also be administered in combination with one or more other therapies (e.g., radiation therapy, immunotherapy). In some embodiments (for example, wherein the cancer is FLT3 inhibitor-resistant AML), the additional therapy is a FLT3 inhibitor (e.g., sunitinib, sorafenib, midostaurin, lestaurtinib, ponatinib, crenolanib, quizartinib, gilteritinib).

In another aspect, the present invention provides a method of determining the prognosis of a subject with a cancer, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein the cancer is not CLL, and wherein a reduction in the level of the miR gene product relative to a threshold level, an absence of a detectable level of the miR gene product, or an allelic loss of the miR gene, in the sample from the subject, is indicative of the subject having a worse prognosis.

In another aspect, the present invention provides a method of determining the prognosis of a subject with a cancer, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying the subject as one who has increased risk of poor prognosis when there is a reduction in the level of the miR gene product relative to a threshold level, an absence of a detectable level of the miR gene product, or an allelic loss of the miR gene, in the sample from the subject,
wherein the cancer is not CLL.

In some embodiments, the method determines whether a subject with a MDS has, or is at risk of developing, an AML. In some embodiments, the method determines whether a subject with a chronic phase CML has, or is at risk of developing, a BC.

In another aspect, the present invention provides a method of determining the prognosis of a subject with a MDS, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, is indicative of the subject either having, or being at risk of developing, an AML.

In another aspect, the present invention provides a method of determining the prognosis of a subject with a MDS, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying the subject as either having, or being at risk of developing, an AML, when there is a reduction in the level of the miR gene product relative to a threshold level, an absence of a detectable level of the miR gene product, or an allelic loss of the miR gene, in the sample from the subject.

In another aspect, the present invention provides a method of determining the prognosis of a subject with a chronic phase CML, comprising determining, in a sample from the subj ect:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, is indicative of the subject either having, or being at risk of developing, a BC.

In another aspect, the present invention provides a method of determining the prognosis of a subject with a chronic phase CML, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying the subject as either having, or being at risk of developing, a BC, when there is a reduction in the level of the miR gene product relative to a threshold level, an absence of a detectable level of the miR gene product, or an allelic loss of the miR gene, in the sample from the subject.

In another aspect, the present invention provides a method of identifying a subject having a cancer as a candidate for a treatment comprising a Bmi-1 inhibitor, a Bcl-2 inhibitor, a ROR1 inhibitor, or a combination thereof, comprising determining, in a sample from the subject:
a) the level of a miR gene product selected from the group consisting of miR-15a, miR-15b, miR-16-1, miR-16-2, and a combination thereof;
b) the status of a miR gene selected from the group consisting of a miR-15a gene, a miR-15b gene, a miR-16-1 gene, a miR-16-2 gene, and a combination thereof; or
c) a combination of a) and b),
wherein the cancer is not CLL, and wherein a reduction in the level of the miR gene product relative to a threshold level, an absence of a detectable level of the miR gene product, or an allelic loss of the miR gene, in the sample from the subject, indicates that the subject is a candidate for a treatment comprising a Bmi-1 inhibitor, a Bcl-2 inhibitor, a ROR1 inhibitor, or a combination thereof.

In another aspect, the present invention provides a method of identifying a subject having a cancer as a candidate for a treatment comprising a Bmi-1 inhibitor, a Bcl-2 inhibitor, a ROR1 inhibitor, or a combination thereof, comprising:
a) determining, in a sample from the subject:
   i. the level of a miR gene product selected from the group consisting of miR-15a, miR-15b, miR-16-1, miR-16-2, and a combination thereof;
   ii. the status of a miR gene selected from the group consisting of a miR-15a gene, a miR-15b gene, a miR-16-1 gene, a miR-16-2 gene, and a combination thereof; or
   iii. a combination of a) and b); and
b) identifying the subject as a candidate for the treatment, when there is a reduction in the level of the miR gene product relative to a threshold level, an absence of a detectable level of the miR gene product, or an allelic loss of the miR gene, in the sample from the subject,
wherein the cancer is not CLL.

In some embodiments, the subject has a MDS or an AML. In some embodiments, the subject has a chronic phase CML or a BC.

In another aspect, the present invention provides a method of identifying a subject having a MDS or an AML as a candidate for a treatment comprising a Bcl-2 inhibitor, a ROR1 inhibitor, or both, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bcl-2 inhibitor, a ROR1 inhibitor, or both.

In another aspect, the present invention provides a method of identifying a subject having a MDS or an AML as a candidate for a treatment comprising a Bcl-2 inhibitor, a ROR1 inhibitor, or both, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying the subject as a candidate for the treatment, when there is a reduction in the level of the miR gene product relative to a threshold level, an absence of a detectable level of the miR gene product, or an allelic loss of the miR gene, in the sample from the subject.

In some embodiments, the method further comprises administering an effective amount of a Bcl-2 inhibitor, an effective amount of a ROR1 inhibitor, or both. In some embodiments, the method further comprises administering an effective amount of venetoclax, an effective amount of a monoclonal antibody against ROR-1, or both.

In another aspect, the present invention provides a method of identifying a subject having a chronic phase CML or BC as a candidate for a treatment comprising a Bmi-1 inhibitor, a Bcl-2 inhibitor, a ROR1 inhibitor, or a combination thereof, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bmi-1 inhibitor, a Bcl-2 inhibitor, a ROR1 inhibitor, or a combination thereof.

In another aspect, the present invention provides a method of identifying a subject having a chronic phase CML or BC as a candidate for a treatment comprising a Bmi-1 inhibitor, a Bcl-2 inhibitor, a ROR1 inhibitor, or a combination thereof, comprising:
a) determining, in a sample from the subject:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) identifying the subject as a candidate for the treatment, when there is a reduction in the level of the miR gene product relative to a threshold level, an absence of a detectable level of the miR gene product, or an allelic loss of the miR gene, in the sample from the subject.

In some embodiments, the method further comprises administering an effective amount of a Bmi-1 inhibitor, an effective a Bcl-2 inhibitor, an effective amount of a ROR1 inhibitor, or a combination thereof. In some embodiments, the method further comprises administering an effective amount of venetoclax, an effective amount of a monoclonal antibody against ROR-1, or both.

The subject, the sample, the miR gene product, the method of measuring and determining the level of the miR gene product, and the therapeutic agents are described herein.

In another aspect, the present invention provides a method of stratifying a set of subjects having cancer for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein the cancer is not CLL, and wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bmi-1 inhibitor, a Bcl-2 inhibitor, a ROR1 inhibitor, or a combination thereof.

In another aspect, the present invention provides a method of stratifying a set of subjects having cancer for treatment, comprising:
a) determining, in samples from the set of subjects:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) stratifying the set of patients for treatment according to the individual patients’ level of a miR gene product, status of a miR gene, or a combination thereof,
wherein the cancer is not CLL, and wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bmi-1 inhibitor, a Bcl-2 inhibitor, a ROR1 inhibitor, or a combination thereof.

In some embodiments, the method stratifies a set of subjects having MDS for treatment. In some embodiments, the method stratifies a set of subjects having chronic phase CML for treatment.

In another aspect, the present invention provides a method of stratifying a set of subjects having MDS for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bcl-2 inhibitor, a ROR1 inhibitor, or both.

In another aspect, the present invention provides a method of stratifying a set of subjects having MDS for treatment, comprising:
a) determining, in samples from the set of subjects:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) stratifying the set of patients for treatment according to the individual patients’ level of a miR gene product, status of a miR gene, or a combination thereof,
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bcl-2 inhibitor, a ROR1 inhibitor, or both.

In another aspect, the present invention provides a method of stratifying a set of subjects having chronic phase CML for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bmi-1 inhibitor, a Bcl-2 inhibitor, a ROR1 inhibitor, or a combination thereof.

In another aspect, the present invention provides a method of stratifying a set of subjects having chronic phase CML for treatment, comprising:
a) determining, in samples from the set of subjects:
   i. the level of a miR gene product selected from the group consisting of miR-15a, miR-15b, miR-16-1, miR-16-2 and combinations thereof;
   ii. the status of a miR gene selected from the group consisting of a miR-15a gene, a miR-15b gene, a miR-16-1 gene, a miR-16-2 gene and combinations thereof; or
   iii. a combination of a) and b); and
b) stratifying the set of patients for treatment according to the individual patients’ level of a miR gene product, status of a miR gene, or a combination thereof,
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a Bmi-1 inhibitor, a Bcl-2 inhibitor, a ROR1 inhibitor, or a combination thereof.

The subject, the sample, the miR gene product, the method of measuring and determining the level of the miR gene product, and the therapeutic agents are described herein.

In another aspect, the present invention provides a method of treating a subject having a cancer, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b),
wherein the cancer is not CLL.

In some embodiments, the subject has an AML. In some embodiments, the subject has a BC.

In another aspect, the present invention provides a method of treating a subject having an AML, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

In some embodiments, the AML is associated with a loss of miR-15a/16-1 on chromosome 13q14, a loss of miR-15b/16-2 on chromosome 3q25, or a combination thereof. In some embodiments, the subject has an AML characterized by a reduced expression of miR-15a, miR-15b, miR-16-1, miR-16-2 or a combination thereof.

In another aspect, the present invention provides a method of treating a subject having a BC, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

In some embodiments, the BC is associated with a loss of miR-15a/16-1 on chromosome 13q14, a loss of miR-15b/16-2 on chromosome 3q25, or a combination thereof. In some embodiments, the subject has a BC characterized by a reduced expression of miR-15a, miR-15b, miR-16-1, miR-16-2 or a combination thereof.

The subject, the sample, the miR gene product, the method of measuring and determining the level of the miR gene product, and the therapeutic agents are described herein.

“Treating,” as used herein, refers to taking steps to deliver a therapy to a subject, such as a mammal (e.g., a human patient), in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

A therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

In another aspect, the present invention provides a method of treating a subject having a cancer, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).
wherein the cancer is not CLL.

In some embodiments, the subject has a MDS, and treating the subject inhibits transformation of the MDS to an AML. In some embodiments, the subject has a chronic phase CML, and treating the subject inhibits transformation of the chronic phase CML to a BC. In some embodiments, the cancer (e.g., MDS or chronic phase CML) is associated with a loss of miR-15a/16-1 on chromosome 13q14, a loss of miR-15b/16-2 on chromosome 3q25, or a combination thereof. In some embodiments, the subject has a cancer (e.g., a MDS or a chronic phase CML) characterized by a reduced expression of miR-15a, miR-15b, miR-16-1, miR-16-2 or a combination thereof.

In another aspect, the present invention provides a method of treating a subject having a MDS, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

In some embodiments, treating the subject inhibits transformation of the MDS to an AML. In some embodiments, the MDS is associated with a loss of miR-15a/16-1 on chromosome 13q14, a loss of miR-15b/16-2 on chromosome 3q25, or a combination thereof. In some embodiments, the subject has a MDS characterized by a reduced expression of miR-15a, miR-15b, miR-16-1, miR-16-2 or a combination thereof.

In another aspect, the present invention provides a method of treating a subject having a chronic phase CML, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

In some embodiments, treating the subject inhibits transformation of the chronic phase CML to a BC. In some embodiments, the chronic phase CML is associated with a loss of miR-15a/16-1 on chromosome 13q14, a loss of miR-15b/16-2 on chromosome 3q25, or a combination thereof. In some embodiments, the subject has a chronic phase CML characterized by a reduced expression of miR-15a, miR-15b, miR-16-1, miR-16-2 or a combination thereof.

The subject, the sample, the miR gene product, the method of measuring and determining the level of the miR gene product, and the therapeutic agents are described herein.

In another aspect, the present invention provides a method of treating a subject having a cancer, comprising
a) determining, in a sample from the subject:
   i) the level of a miR gene product selected from the group consisting of miR-15a, miR-15b, miR-16-1, miR-16-2 and combinations thereof;
   ii) the status of a miR gene selected from the group consisting of a miR-15a gene, a miR-15b gene, a miR-16-1 gene, a miR-16-2 gene and combinations thereof; or
   iii) a combination of a) and b); and
b) administering to the subject:
   i) one or more agents that increase the expression or activity of a miR gene product selected from the group consisting of miR-15a, miR-15b, miR-16-1, miR-16-2 and combinations thereof;
   ii) one or more agents that that reduce the expression or activity of a target of a miR gene product selected from the group consisting of miR-15a, miR-15b, miR-16-1, miR-16-2 and combinations thereof; or
   iii) a combination thereof,
when there is a reduction in the level of the miR gene product relative to a threshold level, an absence of a detectable level of the miR gene product, or an allelic loss of the miR gene, in the sample from the subject, wherein the cancer is not CLL.

In some embodiments, the subject has a MDS. In some embodiments, the subject has a chronic phase CML.

In another aspect, the present invention provides a method of treating a subject having a MDS, comprising
a) determining, in a sample from the subject:
   i) the level of a miR gene product selected from the group consisting of miR-15a, miR-15b, miR-16-1, miR-16-2 and combinations thereof;
   ii) the status of a miR gene selected from the group consisting of a miR-15a gene, a miR-15b gene, a miR-16-1 gene, a miR-16-2 gene and combinations thereof; or
   iii) a combination of a) and b); and
b) administering to the subject:
   iv) one or more agents that increase the expression or activity of a miR gene product selected from the group consisting of miR-15a, miR-15b, miR-16-1, miR-16-2 and combinations thereof;
   v) one or more agents that that reduce the expression or activity of a target of a miR gene product selected from the group consisting of miR-15a, miR-15b, miR-16-1, miR-16-2 and combinations thereof; or
   vi) a combination thereof,
when there is a reduction in the level of the miR gene product relative to a threshold level, an absence of a detectable level of the miR gene product, or an allelic loss of the miR gene, in the sample from the subject.

In another aspect, the present invention provides a method of treating a subject having a chronic phase CML, comprising
a) determining, in a sample from the subject:
   i) the level of a miR gene product selected from the group consisting of miR-15a, miR-15b, miR-16-1, miR-16-2 and combinations thereof;
   ii) the status of a miR gene selected from the group consisting of a miR-15a gene, a miR-15b gene, a miR-16-1 gene, a miR-16-2 gene and combinations thereof; or
   iii) a combination of a) and b); and
b) administering to the subject:
   i) one or more agents that increase the expression or activity of a miR gene product selected from the group consisting of miR-15a, miR-15b, miR-16-1, miR-16-2 and combinations thereof;
   ii) one or more agents that that reduce the expression or activity of a target of a miR gene product selected from the group consisting of miR-15a, miR-15b, miR-16-1, miR-16-2 and combinations thereof; or
   iii) a combination thereof,
when there is a reduction in the level of the miR gene product relative to a threshold level, an absence of a detectable level of the miR gene product, or an allelic loss of the miR gene, in the sample from the subject.

The subject, the sample, the miR gene product, the method of measuring and determining the level of the miR gene product, and the therapeutic agents are described herein.

In another aspect, the present invention provides a method of treating a subject having a cancer that is characterized by loss of expression of miR-15a, miR-15b, miR-16-1, miR-16-2, or a combination thereof, comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

In certain embodiments, the cancer is a hematological cancer other than a chronic lymphocytic leukemia (CLL), such as a hematological cancer other than a B-CLL. In some embodiments, the cancer is a hematological cancer (e.g., MDS, AML or CML (e.g., chronic phase CML or BC)). In some embodiments, the cancer is a solid tumor. In some embodiments, the solid tumor is selected from bladder cancer, brain cancer, breast cancer, cervical cancer, colon cancer, colorectal cancer, gastric cancer, head/neck cancer, kidney cancer, liver cancer, lung cancer, lymphomas, melanomas, oesophageal cancer, ovarian cancer, pancreatic cancer, prostate cancer, sarcomas and combinations thereof.

In some embodiments, treating the patient having a cancer that is characterized by loss of expression of miR-15a, miR-15b, miR-16-1, miR-16-2, or a combination thereof comprises administering an effective amount (e.g., a therapeutically effective amount) of a Bmi-1 inhibitor, a Bcl-2 inhibitor, a ROR1 inhibitor, or a combination thereof (e.g., a Bcl-2 inhibitor, a ROR1 inhibitor, or both).

In another aspect, the present invention provides a method of preparing a sample that is useful for predicting a likelihood of cancer transformation in a subject, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof,
wherein the cancer is not CLL.

In some embodiments, the cancer is a MDS. In some embodiments, the cancer is a chronic phase CML.

In another aspect, the present invention provides a method of preparing a sample that is useful for predicting a likelihood of a subject of developing an AML, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

In some embodiments, the subject has a MDS.

In another aspect, the present invention provides a method of preparing a sample that is useful for predicting a likelihood of a subject of developing a BC, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

In some embodiments, the subject has a chronic phase CML.

In some embodiments, the method comprises reverse transcribing miR-15a, miR-15b, 16-1 and miR-16-2 in the sample to provide target oligodeoxynucleotides.

As used herein, “target oligodeoxynucleotides” refer to the reverse-transcribed cDNA products of the miRNA.

In some embodiments, the method further comprises amplifying the target oligodeoxynucleotides by a polymerase chain reaction (PCR) prior to the quantification step. Quantitative RT-PCR, and variations thereof, are well known to those of skill in the art. In some embodiments, an internal standard (e.g., a housekeeping gene such as myosin or glyceraldehyde-3-phosphate dehydrogenase (GAPDH)) is used for normalization.

In some embodiments, the method further comprises hybridizing the target oligodeoxynucleotides to a microarray, wherein the microarray comprises one or more probes specific for the miR gene products. Microarrays and methods of microarray analysis are well known to those of skill in the art.

In some embodiments, the method further comprises determining the relative level of the target oligodeoxynucleotides in the sample compared to a threshold. In some embodiments, the threshold is based on a control sample or a reference standard.

In some embodiments, the method further comprises quantifying the target oligodeoxynucleotides in the sample prepared in step b).

In some embodiments, the method further comprises contacting the target oligodeoxynucleotides in the sample prepared in step b) with a probe specifically binds the miRNA.

In some embodiments, the expression levels of two or more miR gene products in the sample are determined simultaneously.

The subject, the sample, the miR gene product and the method of measuring and determining the level of the miR gene product are described herein.

In another aspect, the present invention provides a method of preparing a sample that is useful for detecting a subject having cancer cells susceptible to treatment with a Bmi-1 inhibitor, a Bcl-2 inhibitor, a ROR1 inhibitor, or a combination thereof, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof,
wherein the cancer is not CLL.

In some embodiments, the cancer is a MDS. In some embodiments, the cancer is a chronic phase CML.

In another aspect, the present invention provides a method of preparing a sample that is useful for detecting a subject having AML cells susceptible to treatment with a Bcl-2 inhibitor, a ROR1 inhibitor, or both, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

In some embodiments, the subject has a MDS.

In another aspect, the present invention provides a method of preparing a sample that is useful for detecting a subject having BC cells susceptible to treatment with a Bmi-1 inhibitor, a Bcl-2 inhibitor, a ROR1 inhibitor, or a combination thereof, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

In some embodiments, the subject has a chronic phase CML.

In some embodiments, the method comprises reverse transcribing miR-15a, miR-15b, 16-1 and miR-16-2 in the sample to provide target oligodeoxynucleotides.

In some embodiments, the method further comprises amplifying the target oligodeoxynucleotides by a PCR prior to the quantification step.

In some embodiments, the method further comprises determining the relative level of the target oligodeoxynucleotides in the sample compared to a threshold.

In some embodiments, the method further comprises quantifying the target oligodeoxynucleotides in the sample prepared in step b).

In some embodiments, the method further comprises contacting the target oligodeoxynucleotides in the sample prepared in step b) with a probe specifically binds the miRNA.

The subject, the sample, the miR gene product, the threshold and the method of measuring and determining the level of the miR gene product are described herein.

In another aspect, the present invention provides a method of preparing samples that are useful for stratifying a set of subjects having cancer for treatment, comprising:
a) obtaining or having obtained the samples from the subjects; and
b) reverse transcribing a miRNA from the individual samples to provide target oligodeoxynucleotides, wherein the miRNA comprises miR-15a, miR-15b, miR-16-1, miR-16-2 or a combination thereof,
wherein the cancer is not CLL.

In some embodiments, the cancer is MDS. In some embodiments, the cancer is chronic phase CML.

In another aspect, the present invention provides a method of preparing samples that are useful for stratifying a set of subjects having MDS for treatment, comprising:
a) obtaining or having obtained the samples from the subjects; and
b) reverse transcribing a miRNA from the individual samples to provide target oligodeoxynucleotides, wherein the miRNA comprises miR-15a, miR-15b, miR-16-1, miR-16-2 or a combination thereof.

In another aspect, the present invention provides a method of preparing samples that are useful for stratifying a set of subjects having chronic phase CML for treatment, comprising:
a) obtaining or having obtained the samples from the subjects; and
b) reverse transcribing a miRNA from the individual samples to provide target oligodeoxynucleotides, wherein the miRNA comprises miR-15a, miR-15b, miR-16-1, miR-16-2 or a combination thereof.

In some embodiments, the method comprises reverse transcribing miR-15a, miR-15b, 16-1 and miR-16-2 in the sample to provide target oligodeoxynucleotides.

In some embodiments, the method further comprises amplifying the target oligodeoxynucleotides by a PCR prior to the quantification step.

In some embodiments, the method further comprises determining the relative level of the target oligodeoxynucleotides in the sample relative to a threshold.

In some embodiments, the method further comprises quantifying the target oligodeoxynucleotides in the sample prepared in step b).

In some embodiments, the method further comprises contacting the target oligodeoxynucleotides in the sample prepared in step b) with a probe specifically binds the miRNA.

The subject, the sample, the miR gene product, the threshold and the method of measuring and determining the level of the miR gene product are described herein.

### Exemplification

The most common genetic alteration in CLL is the loss of the miR-15a/16-1 locus at 13q14 (Calin GA, et al., PNAS 99(24):15524-29 (2002)) that occurs in well over 70% of the patients with this disease. Rare mutations have also been observed to occur at 7 nucleotides in the 3′ direction of pre-miR-16-1 in CLL (Calin GA et al., The New Englandjournal of medicine 353(17):1793-1801 (2005)). This mutation affects the microRNA (miRNAs) maturation by compromising the Drosha cleveage complex process (Calin GA et al., The New England journal of medicine 353(17):1793-1801 (2005) and Mayr C & Bartel DP, Cell 138(4):673-84 (2009)) thus resulting in a dramatic downregulation of the mature form of miR-15a/16-1. Further studies have indicated that the DNA region defined by this mutation is critical for the Drosha complex cleavage process recognition of the pri-miRNA transcript (Mayr C & Bartel DP, Cell 138(4):673-84 (2009)). The autoimmune New Zealand Black (NZB) mouse, that develops CLL late in life, similarly to CLL in humans, has a point mutation in the flanking region 3′ of the pre-miR-16-1 that also affects the processing of the precursor by the Drosha complex (Raveche ES, et al., Blood 109(12):5079-86 (2007)). Thus, loss of function of miR-15a/16-1 cluster on chromosome 13q14 leads to the development of CLL both in humans and in mice (Calin GA, et al., PNAS 99(24):15524-29 (2002), Calin GA et al., The New Englandjournal of medicine 353(17):1793-1801 (2005) and Raveche ES, et al., Blood 109(12):5079-86 (2007)). Further evidence that the loss miR-15a/16-1 leads to the development of CLL was provided by the establishment of the miR-15a/16-1 knockout mouse model (Klein U et al.,Cancer cell 17(1):28-40 (2010)) that develops CLL late in life (18 months) with a penetrance of approximately 40%.

In both mice and humans, there are two loci of miR-15/16, the second being at 3q25 in humans. miR-15b/16-2 knockout mice developed CLL with higher penetrance and earlier development than the miR-15a/16-1 KO mice (Lovat F et al., PNAS 112(37):11636-41 (2015)). A miR-15/16 double knockout mouse model, crossbreeding miR-15a/16-1 and miR-15b/16-2 knockout (KO) mice, was generated. Unexpectedly, 77% of the miR-15/16 double knockout mice developed AML much earlier than the CLLs of the miR-15a/16-1 and miR-15b/16-2 single knockout mice, while the remaining 23% of the knockout mice developed B cell lymphomas (Lovat F et al., PNAS 115(51):13069-74 (2018)). Analysis of the developed AMLs revealed overexpression of miR-15/16 direct targets such as Bcl2, Cyclin D1 and Cyclin D2 (Lovat F et al., PNAS 115(51):13069-74 (2018)).

Experimental evidences have shown that acute myeloid leukemia (AML) and myelodysplastic syndromes (MDS) originate from clonal transformed immature hematopoietic cells following multiple genetic and epigenetic changes in hematopoietic stem cells and progenitor cells (Cazzola M et al., Blood 122(25):4021-34 (2013), Pandolfi A et al., Stem cells translational medicine 2(2):143-50 (2013) and_Shiozawa Y et al., Blood 130(24):2642-53 (2017)). Since MDS is considered an initial leukemic clonal stage, the study described herein assessed whether the concomitant loss of expression of the two miR-15/16 loci on chromosome 13 and 3 plays a role in the progression of MDS into AML and in the pathogenesis of AML.

Double knockout of the two miR-15/16 loci in mouse resulted in the development of AML. This result suggested that at least a fraction of human AMLs could be due to a similar mechanism. The role of the two miR-15/16 clusters in 93 MDS patients, divided in 3 subgroups: patients with MDS, patients with MDS before transforming into AML (MDS-T) and patients with AML evolving from MDS (MDS-AML), were analyzed. MiRNA expression, target protein expression, genetic loss and silencing were assessed in 139 AML cases and 14 different AML cell lines. MDS-T and MDS-AML patients show a reduction of the expression of miR-15a/-15b/-16 compared to MDS patients. Each miRNA can be used to significantly predict MDS and MDS-T groups. A reduced expression of miR-15a and/or miR-15b were observed in 79% of primary AMLs. The expression of miR-15a/-15b/-16 significantly stratified AML patients in two prognostic classes. Furthermore, 40% of these cell lines showed a combined loss of the expression of the miR-15a/-15b, and overexpression of their direct or indirect targets. A genetic loss of miR-15a and miR-15b and silencing of these two loci by methylation were identified as potential mechanisms underlying the silencing of the two miR-15/16 loci.

Thus, the studies described herein uncover a potential driver oncogenic role in loss of expression of both miR-15/16 clusters in the progression of MDS into AML and in AML pathogenesis. The stratification of AML patients, based on miR-15/16 expression, as described herein, suggests new approaches to targeted and combination therapies for the treatment of this disease.

### Example 1. Material and Methods (AML and MDS Studies)

### Human Tissue Samples

MDS, MDS-T patients and AML patients evolved from MDS status were obtained from Fondazione IRCCS Policlinico San Matteo, Pavia, Italy. MDS patients were categorized into MDS-T if a progression into AML was recorded during their clinical course (median time to AML evolution 25 mo, range of 5-35) and biological samples available at both stages of MDS and AML, or into MDS if the patients did not progress to AML (median follow-up 64 mo, range of 12-147). Human AML samples (peripheral blood or bone marrow biopsy) were obtained from Princess Margaret Cancer Centre in Toronto, Canada. All participants provided written informed consent approved by the Institutional Review Board at Fondazione IRCCS Policlinico San Matteo and Princess Margaret Cancer Centre. All samples and clinical data were deidentified. The characteristics of these patients are described in Tables 1-3. For additional patient information, see Tables S1 and S2 in Lovat et al., Proc Natl Acad Sci USA 117(22): 12332-40 (2020), the contents of which are incorporated herein in their entirety.

### Cell Culture

AML derived cell lines were purchased from the following suppliers: HL-60, KASUMI-1, KG-1 MV4-11, THP-1, and U-937, were from American Type Culture Collection (ATCC, Manassas, VA); MOLM-13, Mono-Mac-6, OCI-AML2, OCI-AML3, NB-4, and EOL-1 were from Deutsche Sammlung von Mikroorganismen und Zellkulturen (DSMZ, Braunschweig, GERMANY). MEG-01, a CML cell line, was from ATCC. All cell lines were maintained in RPMI1640 medium containing 10% fetal bovine serum, streptomycin and ampicillin.

### RNA and Quantitative Real-Time PCR

Total RNA was isolated from human samples and cell lines using TRIzol (Invitrogen, Carlsbad, CA), following the provided instructions. For qRT-PCR, TaqMan miRNA assays (miR-15a#000389, miR-15b#000390, miR-16#000391, (Thermo Fisher Scientific, Waltham, MA)) were used to detect mature miRNAs. For all RNAs from AML patients, reverse transcription reactions were performed using the TaqMan miRNA Reverse Transcription Kit (Applied Biosystems, Foster City, CA) and a RT primer pool by pooling all miRNA-specific stem-loop primers of interest. To reduce the possible variability of the Ct value for low copy number transcripts and to stretch the low amounts of samples, a pre-amplification step using TaqMan PreAmp Master Mix (Applied Biosystems, Foster City, CA) was performed using a preamplification miRNA-specific probe pool. The pre-amplification PCR conditions consisted of 10 min at 95° C., 2 min at 55° C., 2 min at 72° C., followed by 12 cycles of 15 s at 95° C., 4 min at 60° C. and 10 min at 99.9° C. At the end of the run, the pre-amplification products were diluted 8 times in water. All qRT-PCRs were carried out in triplicate using QuantStudio 12 K Flex System (Thermo Fisher Scientific, Waltham, MA). MiR-423-3p (TaqMan Assay #002626 (Thermo Fisher Scientific, Waltham, MA)) was least variable among the MDS, MDS-T, MDS-AML and AML cells from patients in this study and was used to normalize human AML samples (Liang Y, Ridzon D, Wong L, & Chen C (2007) Characterization of microRNA expression profiles in normal human tissues. BMC genomics 8:166.); RNU44 (TaqMan Assay #00194 (Thermo Fisher Scientific, Waltham, MA)) and RNU48 (TaqMan Assay #001006 (Thermo Fisher Scientific, Waltham, MA)) were used as normalizers for AML derived cell lines cell lines. TaqMan gene expression assays from Thermo Fisher Scientific were used to detect mRNA expression of Ccnd1 (Hs00765553_m1) and Ccnd2 (Hs00153380_m1). GAPDH (Hs02786624_g1) and OAZ1 (Hs00427923_ml) were used as normalizers.

### DNA Isolation and Southern Blot Analysis

Genomic DNAs of AML derived cell lines were prepared using Phenol:Chloroform: Isoamyl Alcohol (Invitrogen, Carlsbad, CA), following the provided instructions. 5 ug aliquots of the genomic DNAs were digested with HindIII (50 unit) at 37° C. for 12 hours, separated in a 0.8% agarose gel, and capillary transferred onto a nitrocellulose membrane (BioRad, Hercules, CA) in the presence of 20X SSC. DNAs on the blot were fixed by baking at 80° C. for 2 hours under vacuum. For the probe preparation, repeat-free DNA fragments spanning miR-15A-16.1, miR-15B-16.2 and TBP were PCR-amplified with Q5 polymerase according to the manufacturer’s instruction (New England Biolabs, Ipswich, MA) and cloned into pGEM-T vector (Promega, Madison, WI). After validation by sequencing, 10 pg aliquot of each recombinant was PCR-amplified and 250 ng aliquot of the product was labeled with ECL Direct Nucleic Acid Labeling and Detection Systems (GE Healthcare, Chicago, IL). Hybridization, washing, and signal generation/detection were carried out according to the system manual. Each after hybridization, genomic blot was treated with 0.5X SSC/0.1% SDS at 55° C. for 30 min to inactivate horseradish peroxidase conjugated to the hybridized probe and was sequentially hybridized with 2 other probes. To quantitate hybridization results, signal intensities of HindIII bands detected on the single blot and by 3 probes were analyzed by ImageJ (NIH) software. Bands intensity of miR-15a/16-1 and miR-15b/16-2 was normalized by dividing their values with TBP signal intensity values.

### DNA Extraction From TRIzol and Copy Number Variation Assay

After RNA extraction from TRIzol, 500 µL BEB (back extraction buffer: 4 M guanidine thiocyanate; 50 mM sodium citrate; 1 M Tris) were added to the phenol phase and interphase per 1 mL of TRIzol used for RNA extraction and mix by inversion for 10 min. Samples were then centrifuged at 12,000 g for 30 min at room temperature. The upper phase was transferred in a new tube and 400 µL isopropanol were added per 1 mL of TRIzol used. Samples were centrifuged at 12,000 g for 15 min at 4° C. The supernatant was removed, and the pellet was washed with 500 µL 70% ethanol per 1 mL of TRIzol used followed by centrifugation at 12,000 g for 15 min at 4° C. Pellet was dissolved in about 200 µL of water.

Copy number variation was performed on 10 ng of genomic DNA. Quantitative real-time polymerase chain reaction (PCR) TaqMan Copy Number Assays were performed using two FAM-dye-labeled custom probes targeting miR-15a and miR-15b (Applied Biosystems, Foster City, CA). TaqMan CNV reactions were performed in triplicate using as reference VIC-dye labeled TERT assay.

### Bisulfite Sequence and 5-Aza-2′-Deoxycytidine Treatment

Genomic DNA from cell lines was isolated using Phenol:Chloroform:Isoamyl Alcohol (Thermo Fisher Scientific, Waltham, MA) protocol and bisulfite conversion was performed using EZ DNA Methylation-Gold Kit (Zymo Research, Irvine, CA). For target CpG island spanning miR-15a and miR-15b, methylation specific primers (MSP) for bisulfite treated DNA were designed using methprim software. Bisulfite-converted genomic DNA was amplified using Hot start ZymoTaq DNA Polymerase (Zymo Research, Irvine, CA). The methylated purified PCR fragments were cloned into pGEMT vector (Promega, Madison, WI) and individual clones were sequenced.

The primers used for amplification of CpG island on miR-15a are (Table 5):

15aMFWD: TTTTGGGGTATTTTATGTTTTAGT (SEQ ID NO: 7);

15aMREV: CCGATAATAACCGTCATCTCG (SEQ ID NO: 8);

15aUFWD: TTTGGGGTATTTTATGTTTTAGTGT (SEQ ID NO: 9);

15aUREV: ACCCAATAATAACCATCATCTCATA (SEQ ID NO: 10).

The primers used for amplification of CpG island on miR-15b are (Table 5):

15bMFWD: AAGGATTCGGAGTCGAAATATC (SEQ ID NO: 11);

15bMREV: GAACAAAACAAAAAATAAAAACGTA (SEQ ID NO: 12);

15bUFWD: GTGAAGGATTTGGAGTTGAAATATT (SEQ ID NO: 13);

15bUREV: CAAACAAAACAAAAAATAAAAACATA (SEQ ID NO: 14).

MEG-01, THP-1 and OCI-AML3 cells were treated with 5 µM 5-Aza-2′-deoxycytidine (5-Aza-dC) (Sigma-Aldrich, St. Louis, MO), whereas U-937 and MV4-11 cells were treated with 2.5 µM 5-Aza-dC. After 4 days of treatments (fresh drug was added every 24 hour), cells were harvested and total RNA was isolated and analyzed for qRT-PCR analysis.

### Western Blot Analysis

AML derived cell lines and AML patients’ cells were lysed with RIPA lysis buffer (Cell Signaling Technology, Danvers, Massachusetts) and proteins concentration was determined by using Bradford assay (BioRad, Hercules, CA), following the manufacturer’s instructions. 30 µg of protein lysate were separated on a Criterion Tris-HCl 4-20% pre-cast gel (BioRad, Hercules, CA), transferred onto a nitrocellulose membrane (HybondC; Amersham PLC, Little Chalfont, United Kingdom). Membranes were incubated with anti-Bcl-2, anti-PI3K p85 and anti-mTOR (Cell Signaling Technology, Danvers, Massachusetts), anti-ROR1 (Novus Biologicals, Littleton, CO), anti-Cyclin D1 (Abcam, Cambridge, United Kingdom), anti-Mcl-1 (Santa Cruz Biotechnology, Inc., Dallas, TX), and anti-β-actin (Sigma-Aldrich, St. Louis, MO) and were revealed using appropriate horseradish peroxidase-conjugated secondary antibodies (GE Healthcare, Chicago, IL) for ECL detection (Denville scientific, Holliston, MA or MilliporeSigma, Burlington, MA). Densitometry plots and signal intensity quantification were obtained using Image J software.

### Flow Cytometry

U-937, MV4-11, THP-1 and MonoMac6 were treated for 48 hrs with different concentration of ABT-199 (Synnovator Inc, Durham, NC) and then stained with Annexin V-FITC and Propidium Iodide (Trevigen, Gaithersburg, MD) following manufacture’s protocol.

### Statistical Analysis

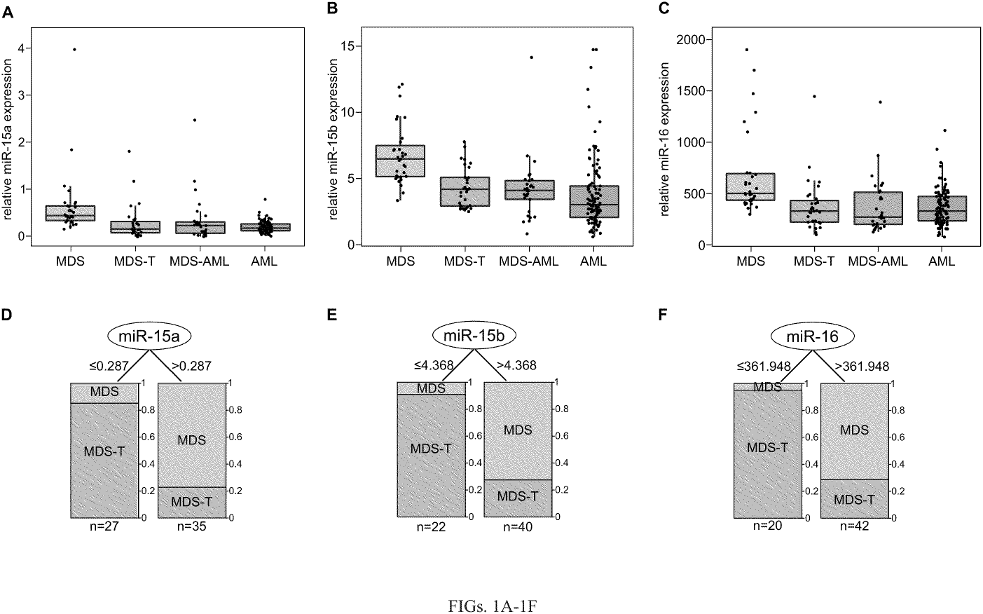

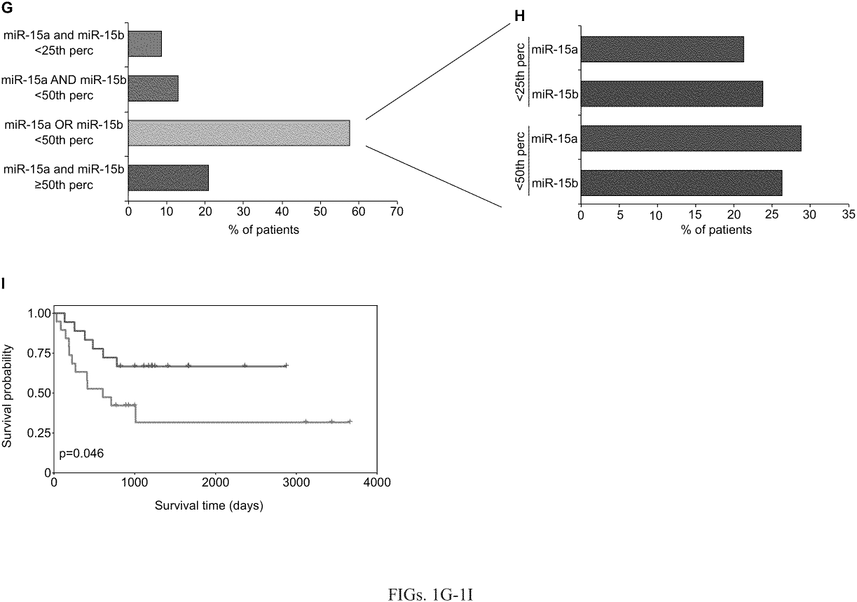

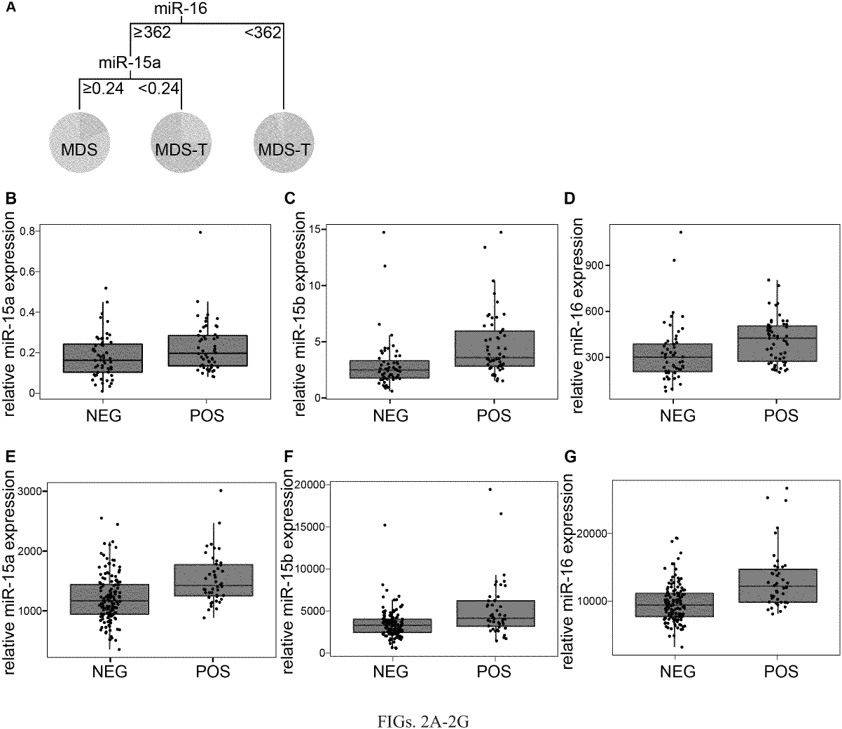

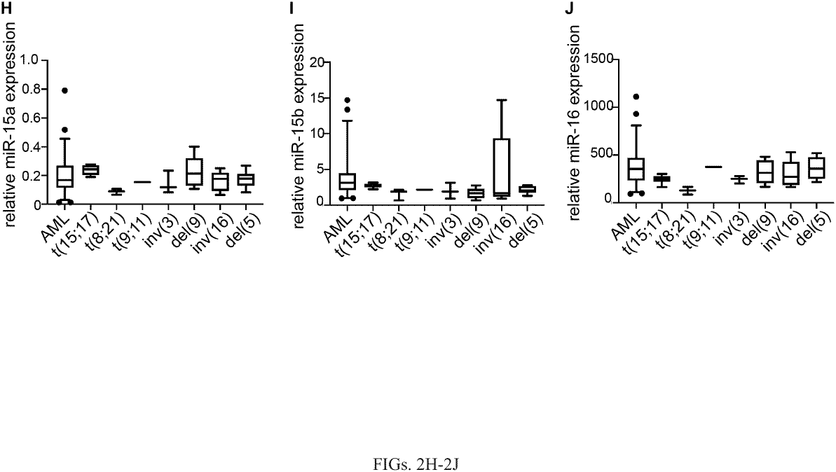

Kruskal-Wallis rank sum test was employed for each non-parametric multivariate analysis present in FIGS. 1A-1C, particularly by using the function kruskal.test function from stats R package. Each p-value was adjusted by using Benjamini Hochberg method employed in the function p.adjusted from stats R package. Wilcoxon rank-sum test was used for each nonparametric pairwise analysis showed in FIGS. 2B-2G, by using the function mannwhitneyu from scipy.stats Python module. The conditional inference tree and decision tree algorithms were applied to classify MDS and MDS-T groups, shown in FIGS. 1D-1F and FIG. 2A, respectively. In particular, FIGS. 1D-1F were generated by using ctree function from party R package, while FIG. 2A was created by using rpart.plot function from rpart.plot R package.

Overall Survival (OS) curves were calculated at last follow up of patients analyzing AML samples by using the Kaplan-Meier method. The two-sided log-rank test was performed taking into consideration all those patients with a combined under-expression (≤50^{th}percentile) and over-expression (>50^{th}percentile) of all three miRNAs (miR-15a, miR-15b and miR-16). The two-sided log-rank tests together with OS curves were performed by using ggsurvplot function from survminer R package. Spearman’s correlation analyses coupled with scatter plots were performed using the function ggscatter from ggpubr R package, considering expressions (2⁻^{ΔCt}values from qRT-PCR) for the CCND1 and CCND2 genes and miR-15a/b and miR-16.

Wilcoxon rank-sum test was used for each non-parametric pairwise analysis showed in FIGS. 2B-2G, by using the function mannwhitneyu from scipy.stats Python module (Python v3.5.2, scipy v1.3.1). Level 3 miRNA expression data of the TCGA Acute Myeloid Leukemia (LAML) cohort were downloaded from GDC data portal. (https://portal.gdc.cancer.gov).

### Example 2 Expression Levels of miR-15a, miR-15b and miR-16 in AML Patients

To establish whether a concomitant loss of expression of the two miR-15/16 loci occurs in human AMLs, the expression of miR-15a, miR-15b, miR-16-1 and miR-16-2 were studied in patients. While the seed region, crucial for mRNA target recognition, is identical in all these four miRNAs, mature miR-15a and miR-15b differ in three nucleotides, while mature miR-16-1 and miR-16-2 are identical and consequently have the same set of targets (hereafter “miR-16”).

Three groups of 31 patients were studied. Group one includes patients with MDS that did not evolve into AML (hereafter “MDS”) (Table 1). Group two includes patients with MDS transforming into AML (hereafter “MDS-T”) (Table 2). Group three includes patients with AML developing from MDS (hereafter “MDS-AML”) (Table 2). The progression of MDS into AML was examined by determining the relative expression of the three miRNAs in these three groups.

As show in FIGS. 1A-1C, the expression of miR-15a (p < 0.001), miR-15b (p < 0.001), and miR-16 (p < 0.001) is progressively reduced in MDS-T and MDS-AML patients compared to MDS patients. The miRNA expressions were further tested in a panel of 139 primary human AML samples derived from two cohorts of 70 and 69 patients (FIGS. 1A-1C and Table 3). The expression of miR-15a, miR-15b and miR-16 is comparable or even reduced in comparison to the MDS-AML patient group. The expression of all three miRNAs is significantly reduced in AML patients compared to MDS group (p < 0.001). For additional patient information, see Tables S1 and S2 in Lovat et al., Proc Natl Acad Sci U S A 117(22): 12332-40 (2020), the contents of which are incorporated herein in their entirety.

Based on their expression, each miRNA, miR-15a, miR-15b and miR-16, can be used to predict MDS and MDS-T groups (in all cases, p < 0.05, see Example 1) (FIGS. 1D-1F). Furthermore, a signature comprising miR-16 and miR-15a, two miRNAs in combination, can also be used to predict MDS and MDS-T groups (see Example 1 for more detail) (FIG. 2A). Specifically, if the value of miR-16 for a sample is higher than the threshold value, 362 (relative expression in 2^{-Δ(ct)} from qRT-PCR), the sample is classified as MDS-T (right pie, accuracy of 95%). If not, but the value of miR-15a for a sample is higher than the threshold value, 0.24 (relative expression in 2^{-Δ(ct)} from qRT-PCR), the sample is also classified as MDS-T (middle pie, accuracy of 75%).

Based on the median expression of miR-15a and miR-15b, AML patients can be divided in two groups. The miR-15a and/or miR-15b low expression group includes 79% of the patients (FIG. 1G, blue, green and orange bars). The miR-15a and miR-15b high expression group includes 21% of the patients (FIG. 1G, red bar). The “low expression group” (79% AML patients) can be divided into three subgroups. The first subgroup includes 8.6% AML patients with miR-15a and miR-15b expression lower than 25^{th}percentile (FIG. 1G, blue bar). The second subgroup includes 12.9% AML patients with miR-15a and miR-15b expression lower than median (50^{th} percentile) (FIG. 1G, green bar). The third subgroup includes 57.6% AML patients with miR-15a or miR-15b expression lower than median (50^{th} percentile) (FIG. 1G, orange bar). These results indicate that a combined loss of expression of both miR-15/16 loci occurs in at least 21.5% of patients with AML (FIG. 1G, blue and green bars). Notably, analyzing the expression of miR-15a and miR-15b of “orange bar” patients in FIG. 1G, any important differences in low (<25^{th} percentile) and medium (<50^{th} percentile) expression of these miRNAs are shown (FIG. 1H).

The Kaplan-Meier estimator was used to analyze the patients’ overall survival (OS), based on miR-15a, miR-15b and miR-16 expression levels. The expression of miR-15a, miR-15b and miR-16 significantly stratified AML patients in two prognostic classes (p = 0.046) (FIG. 11). Patients with a lower expression of all three miRNAs (blue curve) showed a worse outcome compared to patients with a higher expression of these miRNAs (red curve).

The expressions of miR-15a, miR-15b and miR-16 were further analyzed in AML patients with mutated or wild type Nucleophosmin 1 (NPM1) status (FIGS. 2B-2D). A significant upregulation of miR-15a, miR-15b and miR-16 was found in AML patients with mutant NPM1 compared to AML patients with wild type NPM1. This finding is consistent with previous reports (Russ AC et al., Haematologica 96(12):1783-91 (2011) and Garzon R et al., PNAS 105(10):3945-50 (2008)). As a validation dataset, the TCGA database was consulted to verify that miRNAs expression can be used stratify patients based on their NPM1 status (FIGS. 2E-2G). Taken together, these data demonstrate that expression levels of miR-15a, miR-15b and miR-16 can be used to stratify AML patients for diagnosis and prognosis.

In sum, the expression level of miR-15a, miR-15b and miR-16 (mir-16-1 and miR-16-2 are identical and therefore could not be distinguished) were determined in MDS patients who did not progress into AML, in MDS patients before AML transformation (MDS-T) and in AML patients evolved from MDS (MDS-AML). A significant reduction of miR-15a, miR-15b and miR-16 expression occurred during the progression from myelodysplastic syndromes to AML transformation (FIGS. 1A-1C).

When 139 samples from two cohorts of primary AML were tested, most of these patients (79%) show a loss of miR-15/16 expression. Interestingly contemporary loss of expression of both loci (lower than the 50th percentile of their median) occurred in more than 21% of the patients, suggesting that loss of expression of both miR-15/16 loci is a common event in AML. Moreover, as shown in FIG. 1I, the Kaplan Meier analysis indicated a dramatic reduced survival probability in AML patients with a loss of expression of both miRNA loci.

Clonal evolution into AML occurs approximately in 25-30% of MDS patients (Ades L et al., Lancet 383(9936):2239-52 (2014)), and early recognition of patients that are at high risk of progression is crucial to introduce effective treatment strategies (de Witte T et al., Blood 129(13):1753-62 (2017) and Malcovati L et al., Blood 122(17):2943-64 (2013)). Uncovering genetic drivers of clonal progression is therefore instrumental to identify reliable biomarkers for early diagnosis as well as potential therapeutic targets. Interestingly, miR-15/16 cluster is able to stratify MDS patients who will potentially transform in AML (FIGS. 1D-1F and FIG. 2A). Recent studies have shown that gene expression-based classification may improve risk prediction of MDS (Shiozawa Y et al., Blood 130(24):2642-53 (2017)). Thus, miR-15/16 loci may represent robust and reproducible markers for early recognition of patients at high risk of AML evolution.

### Example 3 Mechanisms Underlying Loss of miR-15/16 Expression

### Gene Deletion

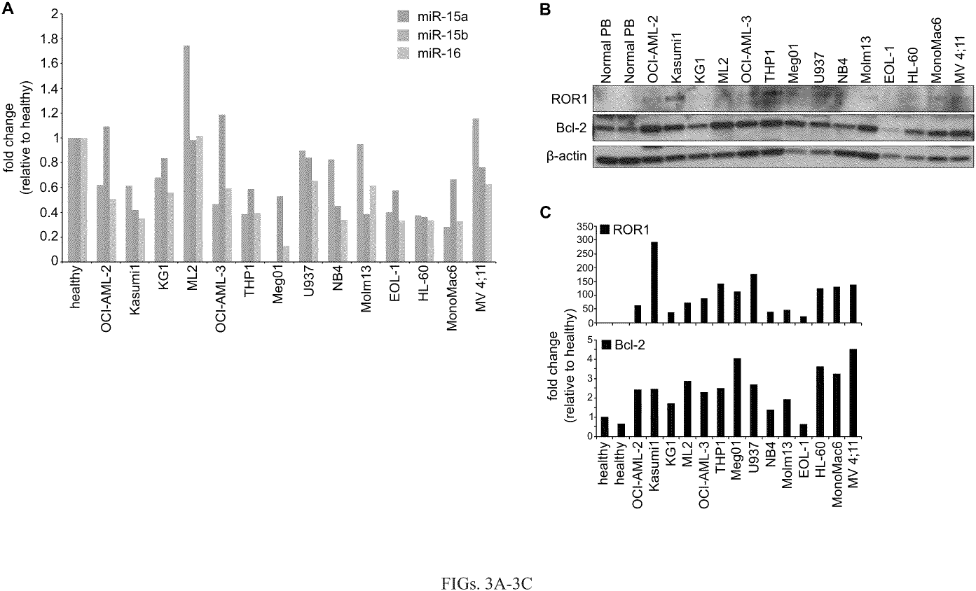

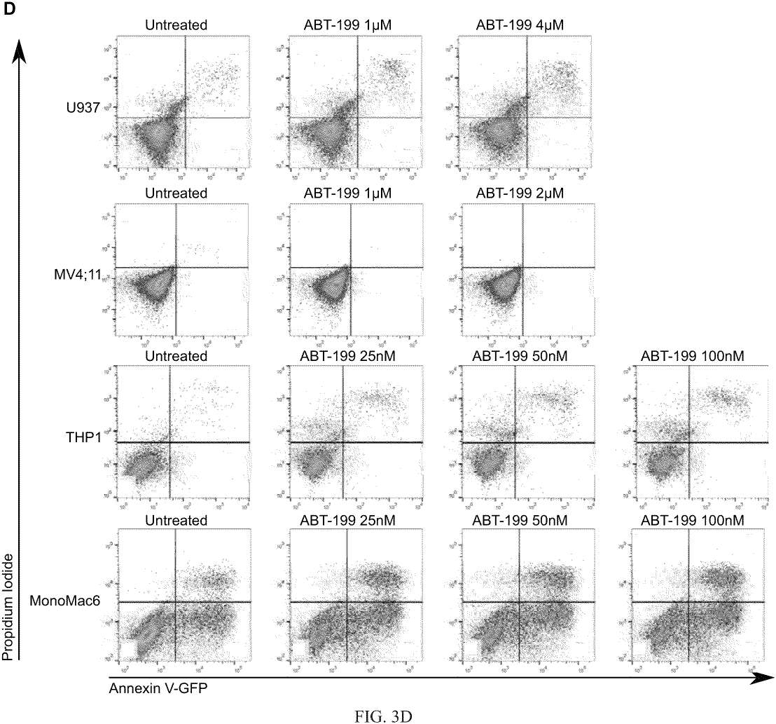

To understand the mechanisms underlying the loss of expression of these miRNAs, a panel of 14 AML-derived cell lines were examined for expression of miR-15/16 clusters. FIG. 3A shows qRT-PCR analysis determining whether most of the AML-derived cell lines have lost expression of miR-15a, miR-15b and miR-16 concomitantly. Approximately 20% of the cell lines showed a combined loss of expression of the two loci. This result supports the finding that the loss of expression of both miR-15/16 loci occurs in at least 21% of AML patients (FIG. 1E, blue and green bars).

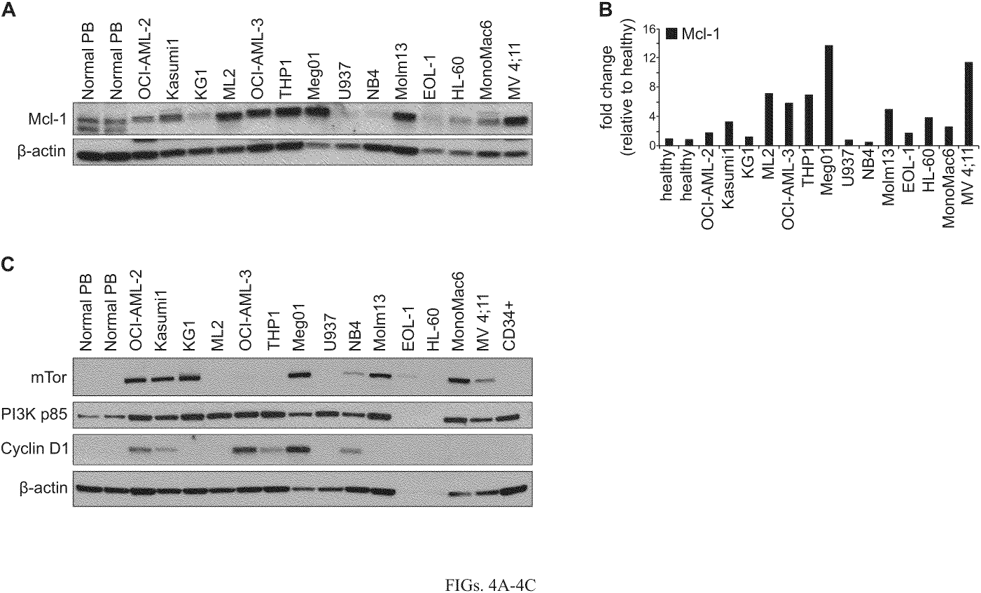

Next, effects of reduced expression of miR-15/16 clusters on the expression of their confirmed direct targets, Bcl-2 and ROR1 (Cimmino A et al., PNAS 102(39):13944-49 (2005) and Rassenti et al., PNAS 114(40):10731-36 (2017)), were tested using Western Blot analysis. Overexpression of Bcl-2 and ROR1 was observed in most AML-derived cell lines (FIG. 3B) compared to normal peripheral blood cells. The observation was confirmed using densitometric analysis (FIG. 3C). Some of the cell lines also overexpressed Cyclin D1 (FIGS. 4C-4D) and Mcl-1 (FIGS. 4A-4B), an indirect target of these miRNAs (Lovat F, et al. (2018), PNAS 115(51):13069-74) that also targets mTOR and PI3K p85 subunit, two genes upstream of Mcl-1 (Janaki Ramaiah M et al., Gene 552(2):255-64 (2014) and Singh Y et al., Journal of immunology 195(12):5667-77 (2015)) (FIGS. 4C-4D).

Since Bcl-2 can be targeted with venetoclax, two cell lines (U-937 and MV4-11) with normal expression of miR-15/16 clusters and two cell lines (THP-1 and MonoMac6) with lower miR-15/16 expression were treated with ABT-199 (venetoclax) for 48 hrs and then tested for apoptosis using Annexin V staining. AML cells with reduced expression of miR-15/16 were more sensitive to ABT-199 treatment compared to AML cells with normal level of miR-15/16 (FIG. 3D).

To determine mechanisms that may be involved in the silencing of miR-15/16 at the different loci, Southern blotting analysis of AML DNAs was performed using miR-15a and miR-15b specific DNA probes. While miR-16-1 and miR-16-2 are immediately adjacent to miR-15a and miR-15b, respectively, being identical, they cannot be distinguished from each other.

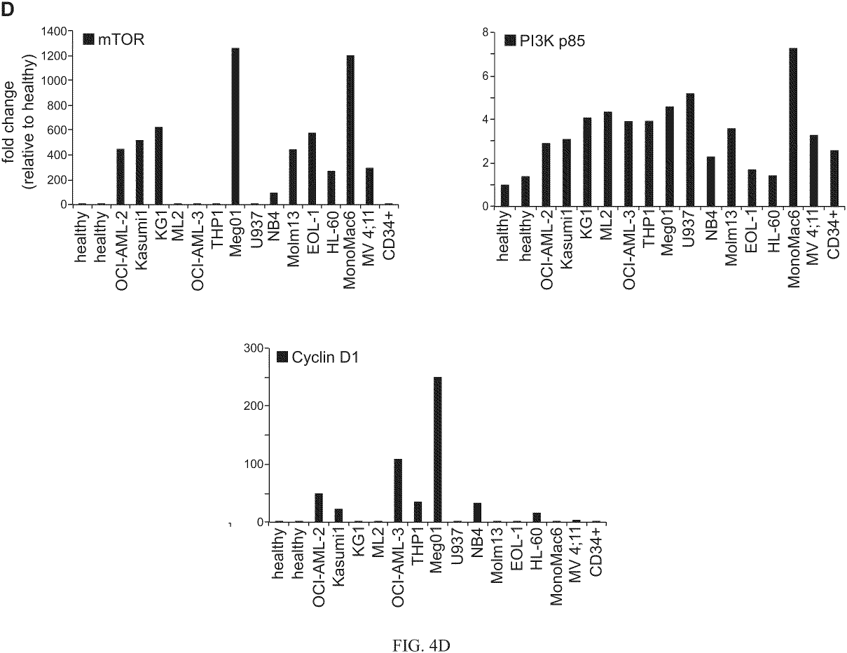

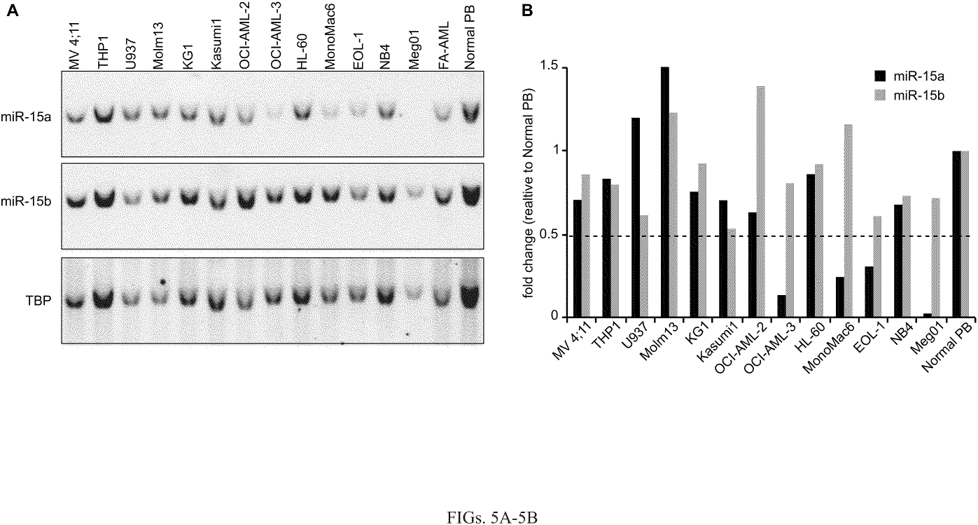

Compared to peripheral blood cells from healthy donors, five out of 13 of the AML-derived cell lines have deletions of copies of miR-15a (FIG. 5A, faded bands indicate loss of a copy the miRNA gene). In MEG-01, a megakaryoblastic leukemia derived cell line, a complete lack of signal indicates a biallelic loss of miR-15a and complete loss of expression of miR-15a (FIG. 5A). The results are consistent with the results obtained by qRT-PCR (FIG. 3A). Deletion of copies of miR-15b was detected in 3 out of 13 cell lines. Densitometric analysis was performed to compare miR-15a and miR15b bands in AML-derived cell lines to normal peripheral blood cells (FIG. 5B).

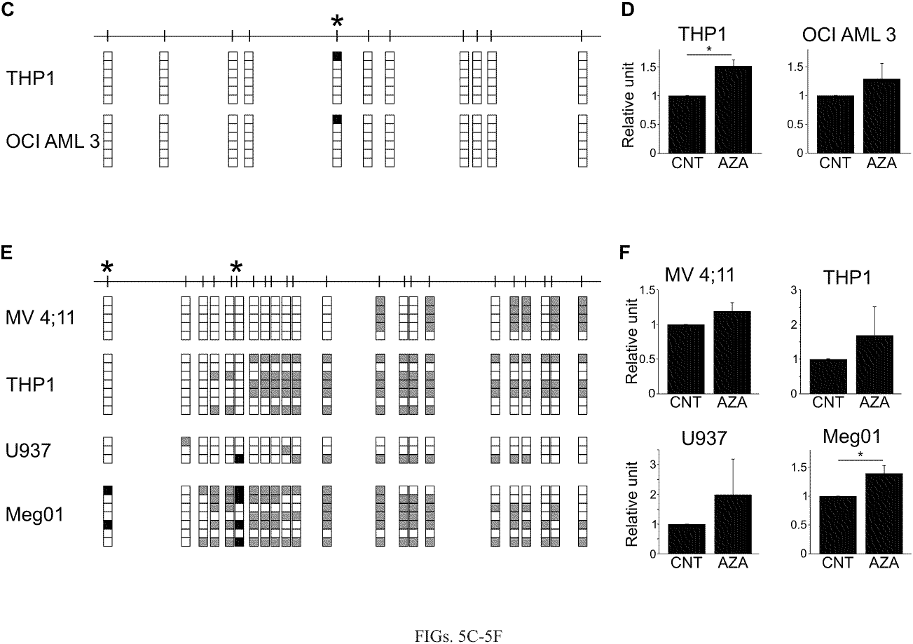

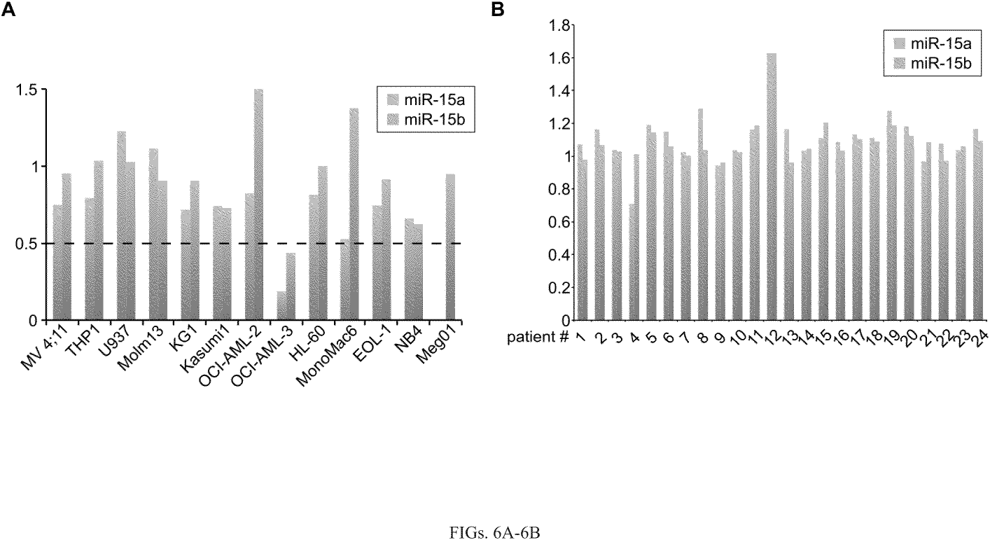

A copy number variation assay was performed using custom probes for detecting miR-15a and miR-15b in genomic DNA from AML cell lines (FIG. 6A) and 24 AML patients (FIG. 6B). The miR-15a deletion was observed in at least one allele in OCI-AML3 and MonoMac6 cell lines, and the miR-15b deletion was observed in at least one allele in Kasumi1 cell line (FIG. 6A). Genomic DNA from 24 AML patients were analyzed, a loss of one copy of miR-15a was found in one patient (FIG. 6B). Thus, gene deletion is one mechanism underlying loss of expression of miR-15/16.

### Methylation Status

Moreover, the methylation status of miR-15a and miR-15b CpG island in AML cell lines was examined by bisulfite sequencing analysis of the genomic DNA region corresponding to the miRNA promoters (miR15a CpG: chr13:50081146-50082272 and miR-15b CpG: chr3:160399225-160401090). The results show a slightly increased methylation of miR-15a (FIG. 5C, black squares) and miR-15b (FIG. 5E, black squares) CpG islands located in the region upstream of the respective miRNAs promoters. In addition, several non-CpG methylated sites in miR-15b CpG island (FIG. 5E, grey squares) were detected, a possible additional mechanism to repress the promoter activity (Inoue S & Oishi M, Gene 348:123-34 (2005) and Malone CS, et al., PNAS 98(18):10404-09 (2001)).

To further confirm the bisulfite sequencing analysis, cell lines were treated with 5-Aza-2′-deoxycytidine (5-Aza-dC), a DNA demethylating agent, to determine whether the miRNA genes may be reactivated by demethylation. In a few cell lines, treatment with 5-Aza-dC resulted in increased expression of the silenced miRNAs, miR-15a (FIG. 5D) and miR-15b (FIG. 5F).

These results suggest that at least two different mechanisms are involved in the loss of expression of miR-15a, miR-15b, and miR-16 in AML. One mechanism is a genetic loss (FIGS. 5A-5B and 6A-6B). The other mechanism is transcription silencing by methylation (FIGS. 5C-5F), reversible by treatment with a demethylating agent.

As shown in FIG. 3B, the loss of miR-15/16 expression in AML cell lines is paralleled by the overexpression of two of the targets of these miRNAs: Bcl-2 and ROR1. The fact that genetic loss and silencing of miR-15/16 causes overexpression of Bcl-2 suggests that dysregulation of Bcl-2 is a driver in the pathogenesis of AML together with downregulation of miR-15/16. Additional potential drivers of disease phenotype can probably be due to the wide spectrum of miR-15a, miR-15b and miR-16 targets. Recent experimental studies provided evidence that blockade of Bcl-2 proteins may efficiently induces apoptosis in progenitor cells of high-risk myelodysplastic syndromes patients (Jilg S et al., Leukemia 30(1):112-23 (2016)). Since Bcl-2 can be targeted with venetoclax, it is logical to predict that AML or MDS patients that have lost miR-15/16 will be sensitive to venetoclax treatment, while patients with normal level of miR-15/16 will not be sensitive to the drug.

### Example 4 Expression of Targets of miR-15/16 Cluster in AML Patients

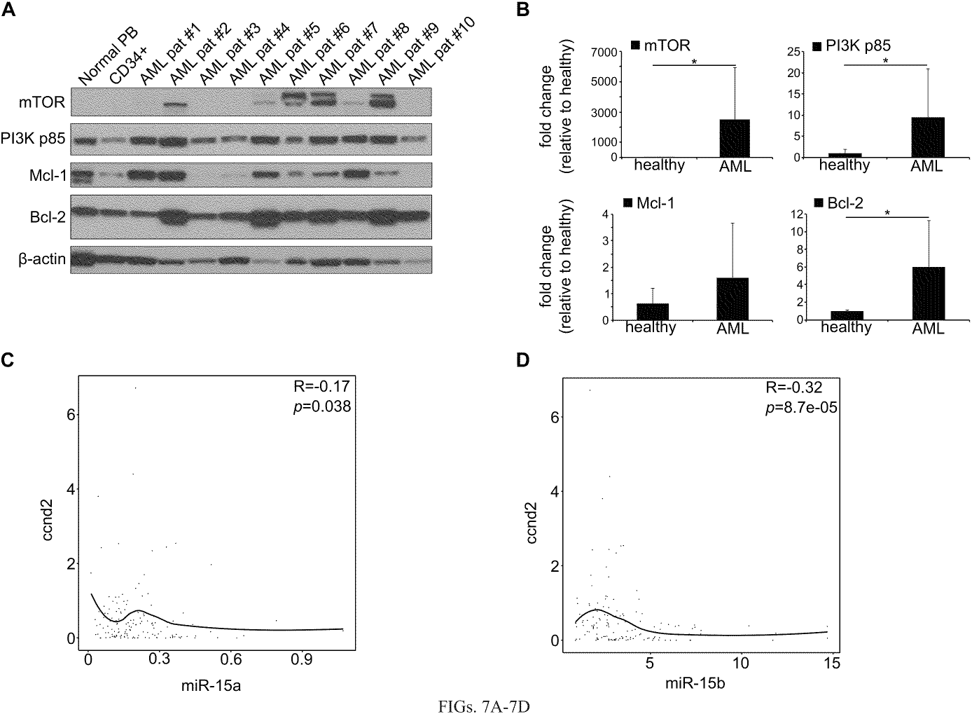

These results indicate that the combined loss of miR-15a, miR-15b, and miR-16 expression occurs frequently in human AMLs and contributes to the development of AML in humans. The expression of known direct or indirect targets of miR-15/16 cluster were tested in AML patients (n = 10) by Western blot analysis. Most of the patients are characterized by an overexpression of Bcl-2, PI3K p85 subunit and mTOR (FIGS. 7A-7B). Furthermore, CCND2 and CCND1 mRNA levels together with miR-15a and miR-15b expression were assessed in the same 139 AML patient cohort (FIGS. 7C-7D and 8A-8B). miR-15a and miR-15b expression levels and CCND2 mRNA expression levels are inversely correlated (FIG. 7C, p = 0.038 and FIG. 7D, p < 0.001). No significant, inverse correlation was observed between miR-15a and miR-15b expression levels and CCND1 mRNA expression levels (FIGS. 8A-8B).

The study disclosed herein suggests that the progression of MDS into AML is driven by a loss of expression of the miR-15/16 loci, thereby providing a robust biological basis for the selection of optimal therapeutic intervention. A combined therapy, based on venetoclax and anti-ROR1 antibody in AML patients stratified by miR-15/16 expression may have several advantages. For example, targeting two different proteins expressed by the same cancer cell avoids the problem of drug resistance.

### Example 5. Materials and Methods (CML Studies)

### Human Tissue Samples

CML unpaired samples were obtained from Princess Margaret Cancer Centre in Toronto, Canada and from MD Anderson Cancer Center in Houston, TX. A total of 39 samples was collected from 22 patients in CP and from 17 patients in BC (Table 6). Paired CML samples were obtained from MD Anderson Cancer Center and The Ohio State University. A total of 22 samples was collected from 11 patients: first set (blood sample or bone marrow aspirates) was collected in CP and second set was collected from the same patient in BC (Table 7). Samples were separated by using Ficoll-Hypaque and viable cells were frozen and stored in liquid nitrogen. This study was carried out under the protocols approved by the Institutional Review Boards of The Ohio State University, the Princess Margaret Cancer Center, and MD Anderson Cancer Center. All samples and clinical data were deidentified. CD34+ from healthy donors were used as the controls.

### RNA and Quantitative Real-Time PCR

Total RNA from CML samples was isolated using TRIzol (Invitrogen, Carlsbad, CA), following the provided instructions. For quantitative real-time PCR (qRT-PCR), TaqMan miRNA assays from Thermo-Fisher (Waltham, MA, miR-15a#000389, miR-15b#000390, miR-16#000391) were used to detect mature miRNAs. qRT-PCR was performed as described by Lovat et al., Proc Natl Acad Sci USA. 117(22):12332-40 (2020). RNU44 (ThermoFisher TaqMan Assay 00194) and RNU48 (ThermoFisher TaqMan Assay 001006) were used as normalizers for CML samples.

### Western Blot Analysis

Due to the low number of cells available per patient sample, especially in BC phase, CML patients’ protein were precipitated after RNA extraction with TRIzol (Invitrogen, Carlsbad, CA) following the protocol described (Simões et al., BMC Genomics 14:181 (2013)). After the last wash with 100% ethanol alcohol (EtOH), the protein pellet was resuspended in 500 µl of 1:1 solution of 1% sodium dodecyl sulfate (SDS) and 8 M urea in Tris HCl 1 M pH 8.0 followed by five cycles of 15-s sonication and 30 s on ice incubation to solubilize the pellet. Then, the sample was concentrated through Amicon column and protein lysate was separated on Criterion Tris HCl 4-20% precast gel (BioRad, Hercules, CA) and transferred onto a nitrocellulose membrane (HybondC, Amersham PLC, Little Chalfont, United Kingdom). Anti-Bcl-2 (Cell Signaling Technologies, Danvers, MA), anti-Bmi-1, and anti-ROR1 (ABclonal Technology, Woburn, MA), anti-GAPDH (GeneTex, Irvine, CA), and anti-β-actin (Sigma-Aldrich, St. Louis, MO) were used to incubate the membrane.

### Statistical Analysis

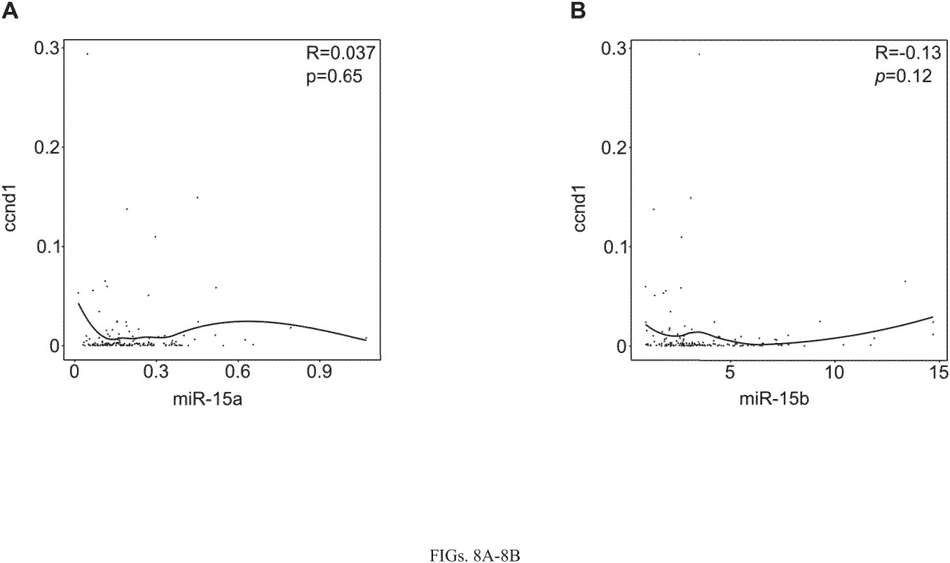

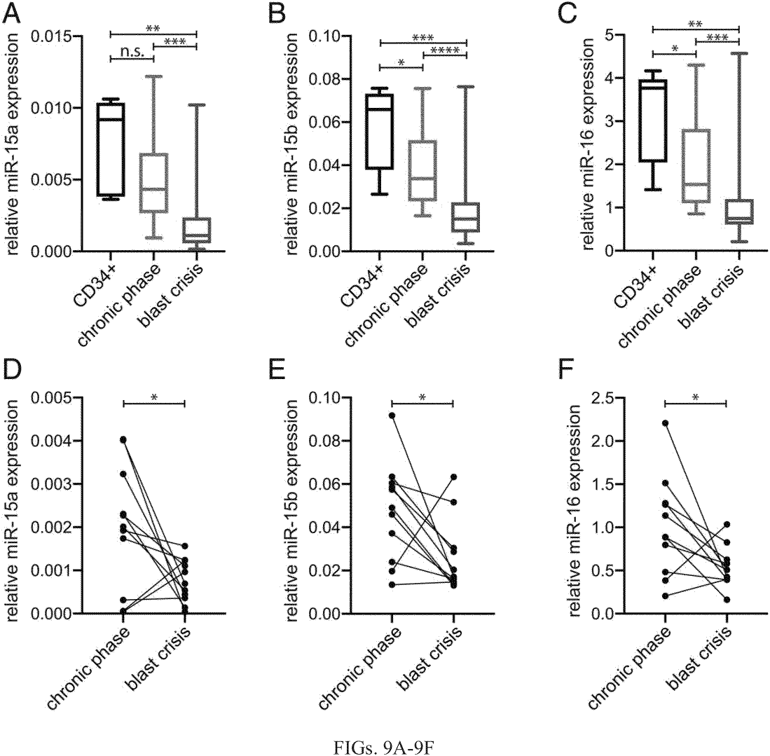

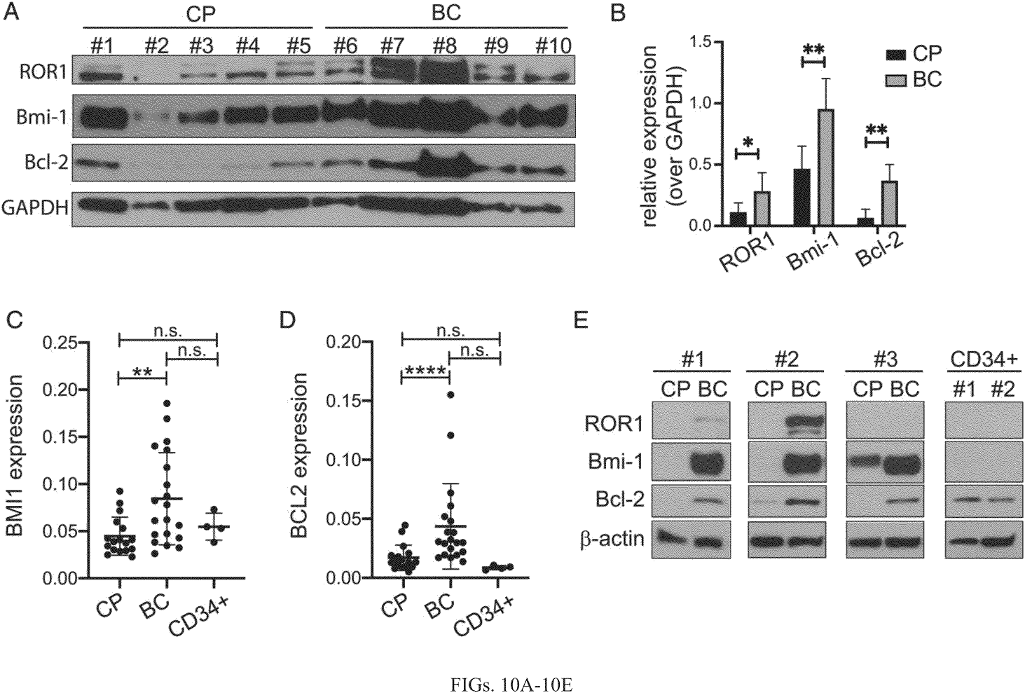

One-way Wilcoxon rank-sum test was used for each unpaired pairwise analysis shown in FIGS. 9A-9C and 10C-10D, while one-way Wilcoxon signed-rank test was applied for each paired analysis shown in FIGS. 9D-9F. For both tests we used the wilcox.test function from the stats R Package (R version 3.5.1). Kruskal-Wallis rank-sum test was employed for each multivariate analysis present in FIGS. 9A-9C and 10C-10D, particularly using the kruskal.test function from the stats R package.

### Example 6 Expression Levels of miR-15a, miR-15b and miR-16 in CML Patients

Mechanisms responsible for the progression of chronic phase CML to BC are not fully understood, although several additional cytogenetic abnormalities, such as trisomy 8, trisomy 19, isochromosome17, and double Philadelphia chromosomes, have been observed in BC CML samples (Wang et al., Blood 127(22):2742-50 (2016)).

MicroRNAs are negative regulators of gene expression by binding to the 3′ UTR of their mRNA targets (Bartel, Cell 136(2):215-33 (2009), Croce, Nat Rev Genet 704-14(2009)). The loss of miR-15/16-1 was observed in the great majority of CLL (~80%). Such loss of expression is due, for the most part, to a deletion of the miR-15/16 locus at chromosome 13q14 and/or epigenetic silencing (Calin et al., Proc Natl Acad Sci USA 99(24):15524-9 (2002), Calin et al., N EnglJMed. 353(17): 1793-801 (2005)). Knockout of the miR-15a/16-1 locus in mice also results in delayed-onset CLL (Klein et al., Cancer Cell 17(1):28-40 (2010)). MiR-15b/16-2, another locus of the miR-15/16 family, was also knocked out, and the KO mice were observed to develop CLL a little earlier and with higher penetrance than the miR-15a/16-1 KO mice (Lovat et al., Proc Natl Acad Sci U S A. 112(37): 11636-41 (2015)). Subsequently, KO mice for both loci: miR-15a/16-1 and miR-15b/16-2 were generated (Lovat et al., Proc Natl Acad Sci USA. 115(51):13069-74 (2018)). Interestingly, it was identified that 77% of the double-KO mice developed AML, while the remaining 23% developed a B-cell lymphoma (Lovat et al., Proc Natl Acad Sci USA. 117(22):12332-40). In addition, ~30%, of MDSs transform into AML (Ades et al., Lancet 383(9936):2239-52 (2014)). Thus, MDS, MDS transforming into AML, AML derived from MDS, and two large cohorts of patients with AML were investigated. It was discovered that loss of expression of both loci of miR-15/16 occurred in the MDS transforming into AML and in a large fraction of AMLs (Lovat et al., Proc Natl Acad Sci USA. 117(22): 12332-40). These losses resulted in overexpression of at least two targets of miR-15/16, BCL2, a driver oncogene, and ROR1, a potential oncogene encoding an embryonic surface antigen (Baskar et al., Clin Cancer Res. 14(2):396-404 (2008), Daneshmanesh et al., Int J Cancer. 123(5):1190-5 (2008)). Thus, loss of miR-15/16 plays an important role not only in the pathogenesis of CLL, but also in the development and progression of a fraction of AMLs and the transformation of MDS into AML.

Since more than 30 years after the discovery of the breakpoint cluster region-Abl tyrosine kinase (BCR/ABL) chimeric oncogene in CML, the cause of progression of CML to BC is still unknown. Here, whether BC progression could be due to the loss of expression of both loci of miR-15/16 was investigated.

First, the expression of miR-15a that maps at 13q14, miR-15b that maps at 3q25, and miR-16 that maps at 13q14 and 3q25 (miR-16-1 and miR-16-2 are identical) was examined in CML patients in chronic phase and BC (Table 6). Normal CD34+ bone marrow cells were used as a control. As shown in FIGS. 9A-9F, chronic-phase CMLs expressed less miR-15a (FIG. 9A), miR-15b (FIG. 9B), and miR-16 (FIG. 9C) compared to normal CD34+ control cells. BC CML cells expressed statistically significantly lower levels of all three microRNAs compared to normal CD34+ cells and to chronic-phase CML cells (FIGS. 9A-9C). The comparison was also carried out in cells from paired chronic phase and BC CML samples (Table 7). Significant decreases of all three microRNAs expression was observed in cells from BC compared to those from chronic phase (FIGS. 9D-9F). These decreases were found in nine out of these 11 pairs for miR-15a (FIG. 9D), miR-15b (FIG. 9E), and miR-16 (FIG. 9F). Interestingly, among these 11 paired samples, two cases from the same patients exhibited very low microRNAs levels in chronic phase, but increased in BC.

Next, protein and transcript expression levels of Bcl-2, ROR1, and Bmi-1, all known targets of miR-15/16 (Bhattacharya et al., Cancer Res. 69(23):9090-5 (2009), Cimmino et al., Proc Natl Acad Sci USA 102(39):13944-9 (2005) and Rassenti et al., Proc Natl Acad Sci USA 114(40):10731-36 (2017)) were determined in five cases of CML in chronic phase and five cases of CML in BC. The expression of all three proteins was more elevated in the five cases of BC compared to the CMLs in chronic phase (with the exception of case 1) as shown by Western blot in FIG. 10A and by its relative quantification (FIG. 10B). A possible explanation of the outlier behavior of case 1 probably lies in the associated clinical information: the cells were collected just 3 months before clinical disease transformation in BC; thus, this CML case was most likely already progressing to BC and protein expression accordingly modulated. Moreover, BMI1 and BCL2 mRNA levels were determined in chronic phase (CP), BC CML patients, and in CD34+ cells from healthy donors. BMI1 and BCL2 expression levels were significantly higher in BC patients compared to CP patients (FIGS. 10C and 10D). No statistically significant differences for BMI1 and BCL2 expression levels were observed in the comparison between CP/BC and C34+ cells from healthy donors. The protein levels of Bcl-2, Bmi-1, and ROR1 were measured in cells from three paired patients in CP and BC and from CD34+ cells from two healthy donors. As shown in FIG. 10E, the expression of ROR1 (undetectable in patient 3 and in CD34+ cells), Bmi-1 (undetectable in CD34+ cells), and Bcl-2 markedly increased when the disease progressed, supporting the finding that progression of CML from chronic to BC is accompanied by higher expression of oncogenic targets of miR-15/16. Moderate to high levels of Bmi-1 were previously detected in some AML patients, especially in M0 subtype of myeloid leukemia (Sawa et al., Int J Hematol 82(1):42-7 (2005)). Interestingly, it has been reported that accelerated and BC CML cells express higher levels of Bmi-1 than CP (Saudy et al., Blood Cells Mol Dis 53(4):194-8 (2014)).

MiR-15/16 target not only BCL2, but also several other oncogenes known to be involved in human and mouse malignancies including BMI1 and MYB, that have been shown to be elevated in CML (Saudy et al., Blood Cells Mol Dis 53(4):194-8 (2014), 22, 23, 24) and AML (Sawa et al., Int J Hematol 82(1):42-7 (2005), 25, 26, 27, 28). It has been shown that a presumptive oncogene ROR1, which is expressed in most CLLs concordantly with BCL2, is also a target of miR-15/16 (Rassenti et al., Proc Natl Acad Sci USA 114(40):10731-36 (2017)). Interestingly the levels of Bmi-1 in BC CMLs appeared to be higher than in CMLs in CP (Mohty et al., Blood 110(1):380-3 (2007)).

From these results, it can be inferred that in most cases of progression from CP into BC CML, miR-15/16 are progressively down-regulated, resulting in overexpression of Bcl-2, ROR1, and Bmi-1, three established oncogenes that promote increased survival and proliferation. Of note, in a minority of two cases, the mechanism of the progression does not seem to involve the enhanced expression of Bcl-2, Bmi-1, and ROR1 since miR-15/16 increased rather than decreased. Without being bound by theory, the deregulation of miR-15/16 expression in the progression from CP to BC is likely due to deletions and/or methylation of miRNAs promoters as previously observed in AML patient samples. The data suggest added therapeutic benefits in the treatment of BC CML by concurrent targeting of these different oncogenes.

In sum, the correlation between the expression of miR-15a/16 and miR-15b/16 with the transition of CML from chronic phase to BC was evaluated. Without being bound by theory, a significant reduction of miR-15a, miR-15b, and miR-16 expression and an overexpression of their targets, such as Bmi-1, ROR1, and Bcl-2, can describe the progression from chronic phase to BC. Thus, targeting different oncogenes activated by the same genetic/epigenetic alteration could represent an important advancement in the treatment of BC CML.

TABLE 1

Characteristics of the MDS patients. These patients did not progress to AML (median follow-up 64 months, range of 12-147)

| Characteristic | Value |
|---|---|
| Age at study entry - year | 69 ± 12.8 |
| Male sex - no. (%) | 17 (55) |
| Female sex - no. (%) | 14 (45) |
| Initial white-cell count (mean) (10⁹ /L) | 5.82 ± 5.95 |
| Initial blasts count (mean) (10⁹ /L) | 7 ± 5.4 |

| | |
|---|---|
| Diagnosis - no. (%) | |
| MDS-RS | 5 (16) |
| MDS-EB1 | 6(19) |
| MDS-EB2 | 12 (39) |
| MDS-CMML-0 | 2 (6.5) |
| MDS-CMML-2 | 1 (3) |
| MDS-MLD | 3 (10) |
| MDS with isolated del(5q) | 2 (6.5) |

TABLE 2

Characteristics of the MDS-T patients with MDS transforming in AML. These patients transformed into AML (MDS-AML) in a period range of 25 months ± 28, range of 5-35

| Characteristic | Value |
|---|---|
| Age at study entry - year | 62 ± 9.8 |
| Male sex - no. (%) | 22 (71) |
| Female sex - no. (%) | 9 (29) |
| Bone Marrow blasts (%) | 8 ± 5.5 |
| Diagnosis - no. (%) | |
| MDS-RS | 4 (13) |
| MDS-EB1 | 6 (19) |
| MDS-EB2 | 12 (39) |
| MDS-CMML-0 | 2 (6) |
| MDS-CMML-2 | 2 (6) |
| MDS-MLD | 3 (10) |
| MDS with isolated del(5q) | 2 (6) |

TABLE 3

Characteristics of the AML form first and second cohorts of patients

| Characteristic | Value of first cohort | Value of second cohort |
|---|---|---|
| Age at study entry - year | 63.5 ± 17.15 | 58 ± 16.47 |
| Male sex - no. (%) | 35 (50) | 28 (40.6) |
| Female sex - no. (%) | 35 (50) | 41 (59.4) |
| Initial white-cell count (mean) (10⁹ /L) | 42.4 ± 84.15 | 54.7 ± 76.72 |
| Initial blasts count (mean) (10⁹ /L) | 24.31 ± 69.94 | 25.54 ± 68.63 |
| Diagnosis - no. (%) | 70 (100) | 69 (100) |
| AML FAB subtype - no. (%) | | |
| AML with minimal differentiation M0 | 2 (3) | 1 (1.5) |
| AML without maturation M1 | 5 (7) | 7 (10.2) |
| AML with maturation M2 | 4 (6) | 5 (7.3) |
| Acute promyelocytic leukemia (APL) M3 | 0 (0) | 7 (10.2) |
| AML M4 | 4 (6) | 12 (17.4) |
| Acute monoblastic or monocytic leukemia M5 | 15 (21) | 12 (17.4) |
| AML | 40 (57) | 25 (36.2) |
| Cytogenetic analysis at diagnosis- no. (%) | | |
| t (8, 21) | 1 (1.5) | 2 (3) |
| t (9, 11) | 1 (1.5) | 0(0) |
| inv (3) | 2 (3) | 1 (1.5) |
| del (9) | 4 (6) | 1 (1.5) |
| t (15, 17) | 0 (0) | 7 (10) |
| inv (16) | 3 (4) | 2 (3) |
| del (5) | 6 (8.5) | 0(0) |
| Mutation at diagnosis - no. (%) | | |
| NPM1 | 24 (34) | 28 (41) |
| FLT3-IDT | 17 (24) | 19 (28) |
| FLT3-TKD | 3 (4) | 2 (3) |

TABLE 4

Human MiRNA Sequences

| SEQ ID NO: | MiRNA Name | MiRNA Sequence |
|---|---|---|
| 1 | precursor miR-15a | CCUUGGAGUAAAGUAGCAGCACAUAAUGGUUUGUGGAUUUUGAAAAGGUGCAGGCCAUAUUGUGCUGCCUCAAAAAUACAAGG |
| 2 | mature miR-15a | UAGCAGCACAUAAUGGUUUGUG |
| 3 | precursor miR-15b | UUGAGGCCUUAAAGUACUGUAGCAGCACAUCAUGGUUUACAUGCUACAGUCAAGAUGCGAAUCAUUAUUUGCUGCUCUAGAAAUUUAAGGAAAUUCAU |
| 4 | mature miR-15b | CGAAUCAUUAUUUGCUGCUCUA |
| 5 | precursor miR-16 | GUCAGCAGUGCCUUAGCAGCACGUAAAUAUUGGCGUUAAGAUUCUAAAAUUAUCUCCAGUAUUAACUGUGCUGCUGAAGUAAGGUUGAC |
| 6 | mature miR-16 | UAGCAGCACGUAAAUAUUGGCG |

TABLE 5

Primer Sequences

| SEQ ID NO: | Primer Name | Primer Sequence |
|---|---|---|
| 7 | 15aMFWD | TTTTGGGGTATTTTATGTTTTAGT |
| 8 | 15aMREV | CCGATAATAACCGTCATCTCG |
| 9 | 15aUFWD | TTTGGGGTATTTTATGTTTTAGTGT |
| 10 | 15aUREV | ACCCAATAATAACCATCATCTCATA |
| 11 | 15bMFWD | AAGGATTCGGAGTCGAAATATC |
| 12 | 15bMREV | GAACAAAACAAAAAATAAAAACGTA |
| 13 | 15bUFWD | GTGAAGGATTTGGAGTTGAAATATT |
| 14 | 15bUREV | CAAACAAAACAAAAAATAAAAACATA |

TABLE 6

Characteristics of CML Patients Included in This Study

| Demographic features | Value |
|---|---|
| Gender, n (%) | |
| Male | 29 (74) |
| Female | 10 (26) |
| Clinical features | |
| Age at diagnosis, median ± SD, y | 50 ± 13.5 |
| Disease phase, n (%) | |
| CP | 17 (44) |
| BC | 22 (56) |
| Initial white cell count, mean ± SD, 10⁹ /L | 93 ± 118.3 |
| Initial blasts count, mean ± SD, 10⁹ /L | 5.6 ± 25.3 |

SD, standard deviation.

TABLE 7

Characteristics of Paired CML Patients Included in This Study

| Demographic features | Value |
|---|---|
| Gender, n (%) | |
| Male | 3 (27) |
| Female | 8 (73) |
| Clinical features | |
| Age at diagnosis, median ± SD, y | 45 ± 17.3 |
| Initial white cell count, mean ± SD, 10⁹ /L | 20.5 ± 197.7 |
| Initial blasts count, mean ± SD, 10⁹ /L | 2.5 ± 26 |

SD, standard deviation.

### REFERENCES

1. De Kouchkovsky I. and Abdul-Hay, M. (2016) Acute myeloid leukemia: a comprehensive review and 2016 update. Blood Cancer Journal 6, e441.

2. Faderl S, Talpaz M, Estrov Z, O′Brien S, Kurzrock R, Kantarjian HM. The biology of chronic myeloid leukemia. N Engl J Med. 1999 Jul 15;341(3):164-72.

3. Kantarjian HM, Keating MJ, Talpaz M, Walters RS, Smith TL, Cork A, McCredie KB, Freireich EJ. Chronic myelogenous leukemia in blast crisis. Analysis of 242 patients. Am J Med. 1987 Sep;83(3):445-54.

4. Sawyers CL. Chronic myeloid leukemia. N Engl J Med. 1999 Apr 29;340(17):1330-40.

5. Radich JP. The Biology of CML blast crisis. Hematology Am Soc Hematol Educ Program. 2007:384-91.

6. Calin GA, Dumitru CD, Shimizu M, Bichi R, Zupo S, Noch E, Aldler H, Rattan S, Keating M, Rai K, Rassenti L, Kipps T, Negrini M, Bullrich F, Croce CM. Frequent deletions and down-regulation of micro- RNA genes miR15 and miR16 at 13q14 in chronic lymphocytic leukemia. Proc Natl Acad Sci U S A. 2002 Nov 26;99(24):15524-9.

7. Calin GA, Ferracin M, Cimmino A, Di Leva G, Shimizu M, Wojcik SE, Iorio MV, Visone R, Sever NI, Fabbri M, Iuliano R, Palumbo T, Pichiorri F, Roldo C, Garzon R, Sevignani C, Rassenti L, Alder H, Volinia S, Liu CG, Kipps TJ, Negrini M, Croce CM. A MicroRNA signature associated with prognosis and progression in chronic lymphocytic leukemia. N Engl J Med. 2005 Oct 27;353(17):1793-801.

8. Mayr C & Bartel DP (2009) Widespread shortening of 3′UTRs by alternative cleavage and polyadenylation activates oncogenes in cancer cells. Cell 138(4):673-684.

9. Raveche ES, et al. (2007) Abnormal microRNA-16 locus with synteny to human 13q14 linked to CLL in NZB mice. Blood 109(12):5079-5086.

10. Klein U, Lia M, Crespo M, Siegel R, Shen Q, Mo T, Ambesi-Impiombato A, Califano A, Migliazza A, Bhagat G, Dalla-Favera R. The DLEU2/miR-15a/16-1 cluster controls B cell proliferation and its deletion leads to chronic lymphocytic leukemia. Cancer Cell. 2010 Jan 19;17(1):28-40.

11. Lovat F, Fassan M, Gasparini P, Rizzotto L, Cascione L, Pizzi M, Vicentini C, Balatti V, Palmieri D, Costinean S, Croce CM. miR-15b/16-2 deletion promotes B-cell malignancies. Proc Natl Acad Sci U S A. 2015 Sep 15;112(37):11636-41.

12. Lovat F, Fassan M, Sacchi D, Ranganathan P, Palamarchuk A, Bill M, Karunasiri M, Gasparini P, Nigita G, Distefano R, Veneziano D, Dorrance AM, Garzon R, Croce CM. Knockout of both miR-15/16 loci induces acute myeloid leukemia. Proc Natl Acad Sci U S A. 2018 Dec 18;115(51):13069-13074.

13. Cimmino A, et al. (2005) miR-15 and miR-16 induce apoptosis by targeting BCL2. Proceedings of the National Academy of Sciences of the United States of America 102(39):13944-13949.

14. Rassenti LZ, Balatti V, Ghia EM, Palamarchuk A, Tomasello L, Fadda P, Pekarsky Y, Widhopf GF 2nd, Kipps TJ, Croce CM. MicroRNA dysregulation to identify therapeutic target combinations for chronic lymphocytic leukemia. Proc Natl Acad Sci U S A. 2017 Oct 3;114(40):10731-10736.

15. Souers AJ, et al. (2013) ABT-199, a potent and selective BCL-2 inhibitor, achieves antitumor activity while sparing platelets. Nature medicine 19(2):202-208.

16. Choi MY, et al. (2015) Pre-clinical Specificity and Safety of UC-961, a First-In-Class Monoclonal Antibody Targeting ROR1. Clinical lymphoma, myeloma & leukemia 15 Suppl:S167-169.

17. Cazzola M, Della Porta MG, & Malcovati L (2013) The genetic basis of myelodysplasia and its clinical relevance. Blood 122(25):4021-4034.

18. Pandolfi A, Barreyro L, & Steidl U (2013) Concise review: preleukemic stem cells: molecular biology and clinical implications of the precursors to leukemia stem cells. Stem cells translational medicine 2(2):143-150.

19. Shiozawa Y, et al. (2017) Gene expression and risk of leukemic transformation in myelodysplasia. Blood 130(24):2642-2653.

20. Russ AC, et al. (2011) Integrative nucleophosmin mutation-associated microRNA and gene expression pattern analysis identifies novel microRNA-target gene interactions in acute myeloid leukemia. Haematologica 96(12):1783-1791.

21. Garzon R, et al. (2008) Distinctive microRNA signature of acute myeloid leukemia bearing cytoplasmic mutated nucleophosmin. Proceedings of the National Academy of Sciences of the United States of America 105(10):3945-3950.

22. Janaki Ramaiah M, et al. (2014) MiR-15/16 complex targets p70S6 kinase 1 and controls cell proliferation in MDA-MB-231 breast cancer cells. Gene 552(2):255-264.

23. Singh Y, Garden OA, Lang F, & Cobb BS (2015) MicroRNA-15b/16 Enhances the Induction of Regulatory T Cells by Regulating the Expression of Rictor and mTOR. Journal of immunology 195(12):5667-5677.

24. Inoue S & Oishi M (2005) Effects of methylation of non-CpG sequence in the promoter region on the expression of human synaptotagmin XI (syt11). Gene 348:123-134.

25. Malone CS, et al. (2001) CmC(A/T)GG DNA methylation in mature B cell lymphoma gene silencing. Proceedings of the National Academy of Sciences of the United States of America 98(18):10404-10409.

26. Ades L, Itzykson R, & Fenaux P (2014) Myelodysplastic syndromes. Lancet 383(9936):2239-2252.

27. de Witte T, et al. (2017) Allogeneic hematopoietic stem cell transplantation for MDS and CMML: recommendations from an international expert panel. Blood 129(13):1753-1762.

28. Malcovati L, et al. (2013) Diagnosis and treatment of primary myelodysplastic syndromes in adults: recommendations from the European LeukemiaNet. Blood 122(17):2943-2964.

29. Jilg S, et al. (2016) Blockade of BCL-2 proteins efficiently induces apoptosis in progenitor cells of high-risk myelodysplastic syndromes patients. Leukemia 30(1):112-123.

30. Liang Y, Ridzon D, Wong L, & Chen C (2007) Characterization of microRNA expression profiles in normal human tissues. BMC genomics 8:166.

31. Lovat F, Nigita G, Distefano R, Nakamura T, Gasparini P, Tomasello L, Fadda P, Ibrahimova N, Catricalà S, Palamarchuk A, Caligiuri MA, Galli A, Malcovati L, Minden MD, Croce CM. Combined loss of function of two different loci of miR-15/16 drives the pathogenesis of acute myeloid leukemia. Proc Natl Acad Sci U S A. 2020 Jun 2;117(22):12332-12340.

32. Simões AE, Pereira DM, Amaral JD, Nunes AF, Gomes SE, Rodrigues PM, Lo AC, D′Hooge R, Steer CJ, Thibodeau SN, Borralho PM, Rodrigues CM. Efficient recovery of proteins from multiple source samples after TRIzol(®) or TRIzol(®)LS RNA extraction and long-term storage. BMC Genomics. 2013 Mar 15;14:181. doi: 10.1186/1471-2164-14-181.

33. Wang W, Cortes JE, Tang G, Khoury JD, Wang S, Bueso-Ramos CE, DiGiuseppe JA, Chen Z, Kantarjian HM, Medeiros LJ, Hu S. Risk stratification of chromosomal abnormalities in chronic myelogenous leukemia in the era of tyrosine kinase inhibitor therapy. Blood. 2016 Jun 2;127(22):2742-50

34. Bartel DP. MicroRNAs: target recognition and regulatory functions. Cell. 2009 Jan 23;136(2):215-33.

35. Croce CM. Causes and consequences of microRNA dysregulation in cancer. Nat Rev Genet. 2009 Oct;10(10):704-14.

36. Ades L, Itzykson R, Fenaux P. Myelodysplastic syndromes. Lancet. 2014 Jun 28;383(9936):2239-52.

37. Baskar S, Kwong KY, Hofer T, Levy JM, Kennedy MG, Lee E, Staudt LM, Wilson WH, Wiestner A, Rader C. Unique cell surface expression of receptor tyrosine kinase ROR1 in human B-cell chronic lymphocytic leukemia. Clin Cancer Res. 2008 Jan 15;14(2):396-404.

38. Daneshmanesh AH, Mikaelsson E, Jeddi-Tehrani M, Bayat AA, Ghods R, Ostadkarampour M, Akhondi M, Lagercrantz S, Larsson C, Osterborg A, Shokri F, Mellstedt H, Rabbani H. Rorl, a cell surface receptor tyrosine kinase is expressed in chronic lymphocytic leukemia and may serve as a putative target for therapy. Int J Cancer. 2008 Sep 1;123(5):1190-5.

39. Bhattacharya R, Nicoloso M, Arvizo R, Wang E, Cortez A, Rossi S, Calin GA, Mukherjee P. MiR-15a and MiR-16 control Bmi-1 expression in ovarian cancer. Cancer Res. 2009 Dec 1;69(23):9090-5.

40. Cimmino A, Calin GA, Fabbri M, Iorio MV, Ferracin M, Shimizu M, Wojcik SE, Aqeilan RI, Zupo S, Dono M, Rassenti L, Alder H, Volinia S, Liu CG, Kipps TJ, Negrini M, Croce CM. miR-15 and miR-16 induce apoptosis by targeting BCL2. Proc Natl Acad Sci U S A. 2005 Sep 27;102(39):13944-9

41. Sawa M, Yamamoto K, Yokozawa T, Kiyoi H, Hishida A, Kajiguchi T, Seto M, Kohno A, Kitamura K, Itoh Y, Asou N, Hamajima N, Emi N, Naoe T. BMI-1 is highly expressed in M0-subtype acute myeloid leukemia. Int J Hematol. 2005 Jul;82(1):42-7.

42. Saudy NS, Fawzy IM, Azmy E, Goda EF, Eneen A, Abdul Salam EM. BMI1 gene expression in myeloid leukemias and its impact on prognosis. Blood Cells Mol Dis. 2014 Dec;53(4):194-8.

43. Mohty M, Yong AS, Szydlo RM, Apperley JF, Melo JV. The polycomb group BMI1 gene is a molecular marker for predicting prognosis of chronic myeloid leukemia. Blood. 2007 Jul 1;110(1):380-3.

44. Lovat F, Gasparini P, Nigita G, Larkin K, Byrd JC, Minden MD, Andreeff M, Carter BZ, Croce CM. Loss of expression of both miR-15/16 loci in CML transition to blast crisis. Proc Natl Acad Sci U S A. 2021 Mar 16;118(11):e2101566118.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of identifying a Myelodysplastic syndrome (MDS) that is likely to transform into an acute myeloid leukemia (AML) in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a MDS that is likely to transform into an AML.

2. A method of identifying a subject having a Myelodysplastic syndrome (MDS) or an acute myeloid leukemia (AML) as a candidate for a treatment comprising a B-cell lymphoma 2 (Bcl-2) inhibitor, a Receptor Tyrosine Kinase Like Orphan Receptor 1 (ROR1) inhibitor, or both, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bcl-2 inhibitor, a ROR1 inhibitor, or both.

3. The method of claim 2, wherein the subject has a MDS.

4. The method of claim 2, wherein the subject has an AML.

5. A method of stratifying a set of subjects having Myelodysplastic syndrome (MDS) for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a B-cell lymphoma 2 (Bcl-2) inhibitor, a Receptor Tyrosine Kinase Like Orphan Receptor 1 (ROR1) inhibitor, or both.

6. The method of any one of claims 1-5, wherein the subject is a human.

7. The method of any one of claims 1-6, wherein the sample comprises bone marrow cells.

8. The method of any one of claims 1-6, wherein the sample comprises a blood sample.

9. The method of any one of claims 1-8, wherein the subject has a loss of miR-15a/16-1 on chromosome 13q14, a loss of miR-15b/16-2 on chromosome 3q25, or both.

10. The method of any one of claims 1-9, wherein the miR gene product is a mature miRNA.

11. The method of any one of claims 1-9, wherein the miR gene product is a pre-cursor miRNA.

12. The method of any one of claims 1-11, wherein the level of the miR gene product is measured using an assay selected from the group consisting of northern blot analysis, in situ hybridization, microarray analysis and quantitative reverse transcriptase polymerase chain reaction.

13. The method of claim 12, wherein the level of the miR gene product is measured using quantitative reverse transcriptase polymerase chain reaction.

14. The method of any one of claims 1-13, wherein the threshold level is the level of the corresponding miR gene product in a control sample or a reference standard.

15. The method of claim 14, wherein:
a) the miR gene product is miR-15a, and the level of miR-15a in the sample is less than about 50% of the threshold miR-15a level;
b) the miR gene product is miR-15b, and the level of miR-15b in the sample is less than about 50% of the threshold miR-15b level;
c) the miR gene product is miR-16, and the level of miR-16 in the sample is less than about 50% of the threshold miR-16 level; or
d) a combination thereof.

16. The method of any one of claims 1-15, comprising determining the levels of each of miR-15a, miR-15b and miR-16 gene products in the sample.

17. The method of any one of claims 1-16, further comprising administering to the subject:
a) one or more agents that increase the expression or activity of the miR gene product;
b) one or more agents that that reduce the expression or activity of a target of the miR gene product; or
c) a combination of a) and b),
when there is a reduction in the level of the miR gene product relative to the threshold level, an absence of a detectable level of the miR gene product, or an allelic loss of the miR gene, in the sample from the subject.

18. The method of claim 17, comprising administering an effective amount of one or more agents that increase the expression or activity of miR-15a, miR-15b, miR-16-1, miR-16-2, or a combination thereof.

19. The method of claim 18, comprising administering an effective amount of one or more agents that increase the expression or activity of miR-15a, miR-15b, miR-16-1 and miR-16-2.

20. The method of claim 17, comprising administering an effective amount of one or more agents that reduce the expression or activity of a target selected from the group consisting of B-cell lymphoma 2 (Bcl-2), Receptor Tyrosine Kinase Like Orphan Receptor 1 (ROR1), Cyclin D1, myeloid cell leukemia 1 (Mcl-1) and combinations thereof.

21. The method of claim 20, comprising administering an effective amount of a Bcl-2 inhibitor.

22. The method of claim 21, wherein the Bcl-2 inhibitor comprises venetoclax, or a pharmaceutically acceptable salt thereof.

23. The method of claim 20, comprising administering an effective amount of a ROR-1 inhibitor.

24. The method of claim 23, wherein the ROR-1 inhibitor comprises a monoclonal antibody against ROR-1.

25. The method of claim 20, comprising administering an effective amount of a Bcl-2 inhibitor and an effective amount of a ROR1 inhibitor.

26. The method of claim 25, comprising administering an effective amount of venetoclax and an effective amount of a monoclonal antibody against ROR-1.

27. A method of treating a subject having an acute myeloid leukemia (AML), comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

28. A method of treating a subject having a Myelodysplastic syndrome (MDS), comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

29. The method of claim 28, wherein treating the subject inhibits transformation of the MDS to an AML.

30. The method of claim 27, 28 or 29, wherein the subject is a human.

31. The method of claim 27, wherein the AML is associated with a loss of miR-15a/16-1 on chromosome 13q14, a loss of miR-15b/16-2 on chromosome 3q25, or a combination thereof.

32. The method of claim 27, wherein the subject has an AML characterized by reduced expression of miR-15a, miR-15b, miR-16-1, miR-16-2 or a combination thereof.

33. The method of claim 28, wherein the MDS is associated with a loss of miR-15a/16-1 on chromosome 13q14, a loss of miR-15b/16-2 on chromosome 3q25, or a combination thereof.

34. The method of claim 28, wherein the subject has a MDS characterized by reduced expression of miR-15a, miR-15b, miR-16-1, miR-16-2 or a combination thereof.

35. The method of any one of claims 27-34, comprising administering to the subject an effective amount of one or more agents that increase the expression or activity of the miR gene.

36. The method of any one of claims 27-35, comprising administering an effective amount of one or more agents that reduce the expression or activity of a target of the miR gene selected from the group consisting of B-cell lymphoma 2 (Bcl-2), Receptor Tyrosine Kinase Like Orphan Receptor 1 (ROR1), Cyclin D1, myeloid cell leukemia 1 (Mcl-1) and combinations thereof.

37. The method of claim 36, wherein the one or more agents that reduce the expression or activity of the target of the miR gene comprises a Bcl-2 inhibitor.

38. The method of claim 37, wherein the Bcl-2 inhibitor comprises venetoclax, or a pharmaceutically acceptable salt thereof.

39. The method of claim 36, wherein the one or more agents that reduce the expression or activity of a target of the miR gene comprises a ROR1 inhibitor.

40. The method of claim 39, wherein the ROR-1 inhibitor comprises a monoclonal antibody against ROR-1.

41. The method of claim 36, comprising administering an effective amount of a Bcl-2 inhibitor and an effective amount of a ROR1 inhibitor.

42. The method of claim 41, comprising administering an effective amount of venetoclax and an effective amount of a monoclonal antibody against ROR-1.

43. A method of preparing a sample that is useful for predicting a likelihood of a subject of developing an acute myeloid leukemia (AML), comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

44. A method of preparing a sample that is useful for detecting a subject having acute myeloid leukemia (AML) cells susceptible to treatment with a B-cell lymphoma 2 (Bcl-2) inhibitor, a Receptor Tyrosine Kinase Like Orphan Receptor 1 (ROR1) inhibitor, or both, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

45. The method of claim 43 or 44, wherein the subject has a Myelodysplastic syndrome (MDS).

46. A method of preparing samples that are useful for stratifying a set of subjects having Myelodysplastic syndrome (MDS) for treatment, comprising:
a) obtaining or having obtained the samples from the subjects; and
b) reverse transcribing a miRNA from the individual samples to provide target oligodeoxynucleotides, wherein the miRNA is miR-15a, miR-15b, miR-16-1, miR-16-2 or a combination thereof.

47. The method of any one of claims 43-46, wherein the subject is a human.

48. The method of any one of claims 43-47, wherein the sample comprises bone marrow cells.

49. The method of any one of claims 43-47, wherein the sample comprises a blood sample.

50. The method of any one of claims 43-49, further comprising quantifying the target oligodeoxynucleotides in the sample prepared in step b).

51. The method of any one of claims 43-50, further comprising comparing the level of the target oligodeoxynucleotides in the sample with a threshold level.

52. The method of claim 51, wherein threshold level is the level of target oligodeoxynucleotides obtained from reverse transcription of a corresponding miR gene product in a control sample or a reference standard.

53. The method of claim 51 or 52, wherein:
a) the miR gene product is miR-15a, and the level of miR-15a in the sample is less than about 50% of the threshold miR-15a level;
b) the miR gene product is miR-15b, and the level of miR-15b in the sample is less than about 50% of the threshold miR-15b level;
c) the miR gene product is miR-16, and the level of miR-16 in the sample is less than about 50% of the threshold miR-16 level; or
d) a combination thereof.

54. The method of any one of claims 43-53, comprising reverse transcribing miR-15a, miR-15b, 16-1 and miR-16-2 in the sample to provide the target oligodeoxynucleotides.

55. A method of identifying a chronic phase chronic myeloid leukemia (CML) that is likely to transform into a blast crisis (BC) in a subject, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject has a chronic phase CML that is likely to transform into a BC.

56. A method of identifying a subject having a chronic phase chronic myeloid leukemia (CML) or a blast crisis (BC) as a candidate for a treatment comprising a B cell-specific Moloney murine leukemia virus integration site 1 (Bmi-1) inhibitor, B-cell lymphoma 2 (Bcl-2) inhibitor, a Receptor Tyrosine Kinase Like Orphan Receptor 1 (ROR1) inhibitor, or a combination thereof, comprising determining, in a sample from the subject:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the sample from the subject, indicates that the subject is a candidate for a treatment comprising a Bmi-1 inhibitor, a Bcl-2 inhibitor, a ROR1 inhibitor, or a combination thereof.

57. The method of claim 56, wherein the subject has a chronic phase CML.

58. The method of claim 56, wherein the subject has a CML.

59. A method of stratifying a set of subjects having chronic myeloid leukemia (CML) for treatment, comprising determining, in samples from the set of subjects:
a) the level of a miR gene product selected from the group consisting of miR-15a, miR-15b, miR-16-1, miR-16-2 and combinations thereof;
b) the status of a miR gene selected from the group consisting of a miR-15a gene, a miR-15b gene, a miR-16-1 gene, a miR-16-2 gene and combinations thereof; or
c) a combination of a) and b),
wherein a reduction in the level of the miR gene product relative to a threshold level, an absence of a detectable level of the miR gene product, or an allelic loss of the miR gene, in the samples from a subset of subjects, identifies the subset of subjects who are candidates for a treatment comprising a B cell-specific Moloney murine leukemia virus integration site 1 (Bmi-1) inhibitor, a B-cell lymphoma 2 (Bcl-2) inhibitor, a Receptor Tyrosine Kinase Like Orphan Receptor 1 (ROR1) inhibitor, or a combination thereof.

60. The method of any one of claims 55-59, wherein the subject is a human.

61. The method of any one of claims 55-60, wherein the sample comprises bone marrow cells.

62. The method of any one of claims 55-60, wherein the sample comprises a blood sample.

63. The method of any one of claims 55-62, wherein the subject has a loss of miR-15a/16-1 on chromosome 13q14, a loss of miR-15b/16-2 on chromosome 3q25, or both.

64. The method of any one of claims 55-63, wherein the miR gene product is a mature miRNA.

65. The method of any one of claims 55-63, wherein the miR gene product is a pre-cursor miRNA.

66. The method of any one of claims 55-65, wherein the level of the miR gene product is measured using an assay selected from the group consisting of northern blot analysis, in situ hybridization, microarray analysis and quantitative reverse transcriptase polymerase chain reaction.

67. The method of claim 66, wherein the level of the miR gene product is measured using quantitative reverse transcriptase polymerase chain reaction.

68. The method of any one of claims 55-67, wherein the threshold level is the level of the corresponding miR gene product in a control sample or a reference standard.

69. The method of claim 68, wherein:
a) the miR gene product is miR-15a, and the level of miR-15a in the sample is less than about 50% of the threshold miR-15a level;
b) the miR gene product is miR-15b, and the level of miR-15b in the sample is less than about 50% of the threshold miR-15b level;
c) the miR gene product is miR-16, and the level of miR-16 in the sample is less than about 50% of the threshold miR-16 level; or
d) a combination thereof.

70. The method of any one of claims 55-69, comprising determining the levels of each of miR-15a, miR-15b and miR-16 gene products in the sample.

71. The method of any one of claims 55-70, further comprising administering to the subject:
a) one or more agents that increase the expression or activity of the miR gene product;
b) one or more agents that that reduce the expression or activity of a target of the miR gene product; or
c) a combination of a) and b),
when there is a reduction in the level of the miR gene product relative to the threshold level, an absence of a detectable level of the miR gene product, or an allelic loss of the miR gene, in the sample from the subject.

72. The method of claim 71, comprising administering an effective amount of one or more agents that increase the expression or activity of miR-15a, miR-15b, miR-16-1, miR-16-2, or a combination thereof.

73. The method of claim 72, comprising administering an effective amount of one or more agents that increase the expression or activity of miR-15a, miR-15b, miR-16-1 and miR-16-2.

74. The method of claim 71, comprising administering an effective amount of one or more agents that reduce the expression or activity of a target selected from the group consisting of B cell-specific Moloney murine leukemia virus integration site 1 (Bmi-1), B-cell lymphoma 2 (Bcl-2), Receptor Tyrosine Kinase Like Orphan Receptor 1 (ROR1), Cyclin D1, myeloid cell leukemia 1 (Mcl-1) and combinations thereof.

75. The method of claim 74, comprising administering an effective amount of a Bcl-2 inhibitor.

76. The method of claim 75, wherein the Bcl-2 inhibitor comprises venetoclax, or a pharmaceutically acceptable salt thereof.

77. The method of claim 74, comprising administering an effective amount of a ROR-1 inhibitor.

78. The method of claim 77, wherein the ROR-1 inhibitor comprises a monoclonal antibody against ROR-1.

79. The method of claim 74, comprising administering an effective amount of a Bmi-1 inhibitor.

80. The method of claim 79, wherein the Bmi-1 inhibitor comprises artemisinin, PRT4165, PTC209, PTC596 or QW24, or a pharmaceutically acceptable salt thereof.

81. A method of treating a subject having a blast crisis (BC), comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

82. A method of treating a subject having a chronic phase chronic myeloid leukemia (CML), comprising administering to the subject an effective amount of:
a) one or more agents that increase the expression or activity of a miR gene product selected from the group consisting of miR-15a, miR-15b, miR-16-1, miR-16-2 and combinations thereof;
b) one or more agents that that reduce the expression or activity of a target of a miR gene product selected from the group consisting of miR-15a, miR-15b, miR-16-1, miR-16-2 and combinations thereof; or
c) a combination of a) and b).

83. The method of claim 82, wherein treating the subject inhibits transformation of the chronic phase CML to a blast crisis (BC).

84. The method of claim 81, 82 or 83, wherein the subject is a human.

85. The method of claim 81, wherein the BC is associated with a loss of miR-15a/16-1 on chromosome 13q14, a loss of miR-15b/16-2 on chromosome 3q25, or a combination thereof.

86. The method of claim 81, wherein the subject has a BC characterized by reduced expression of miR-15a, miR-15b, miR-16-1, miR-16-2 or a combination thereof.

87. The method of claim 82, wherein the chronic phase CML is associated with a loss of miR-15a/16-1 on chromosome 13q14, a loss of miR-15b/16-2 on chromosome 3q25, or a combination thereof.

88. The method of claim 82, wherein the subject has a chronic phase CML characterized by reduced expression of miR-15a, miR-15b, miR-16-1, miR-16-2 or a combination thereof.

89. The method of any one of claims 81-88, comprising administering to the subject an effective amount of one or more agents that increase the expression or activity of the miR gene.

90. The method of any one of claims 81-89, comprising administering an effective amount of one or more agents that reduce the expression or activity of a target of the miR gene selected from the group consisting of B cell-specific Moloney murine leukemia virus integration site 1 (Bmi-1), B-cell lymphoma 2 (Bcl-2), Receptor Tyrosine Kinase Like Orphan Receptor 1 (ROR1), Cyclin D1, myeloid cell leukemia 1 (Mcl-1) and combinations thereof.

91. The method of claim 90, wherein the one or more agents that reduce the expression or activity of the target of the miR gene comprises a Bcl-2 inhibitor.

92. The method of claim 91, wherein the Bcl-2 inhibitor comprises venetoclax, or a pharmaceutically acceptable salt thereof.

93. The method of claim 90, wherein the one or more agents that reduce the expression or activity of a target of the miR gene comprises a ROR1 inhibitor.

94. The method of claim 93, wherein the ROR-1 inhibitor comprises a monoclonal antibody against ROR-1.

95. The method of claim 90, comprising administering an effective amount of a Bmi-1 inhibitor.

96. The method of claim 95, wherein the Bmi-1 inhibitor comprises artemisinin, PRT4165, PTC209, PTC596 or QW24, or a pharmaceutically acceptable salt thereof.

97. A method of preparing a sample that is useful for predicting a likelihood of a subject of developing a blast crisis (BC), comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

98. A method of preparing a sample that is useful for detecting a subject having blast crisis (BC) cells susceptible to treatment with a B cell-specific Moloney murine leukemia virus integration site 1 (Bmi-1) inhibitor, a B-cell lymphoma 2 (Bcl-2) inhibitor, a Receptor Tyrosine Kinase Like Orphan Receptor 1 (ROR1) inhibitor, or a combination thereof, comprising:
a) obtaining or having obtained the sample from the subject; and
b) reverse transcribing a miRNA from the sample to provide target oligodeoxynucleotides, wherein the miRNA is miR-15a, miR-15b, miR-16-1, miR-16-2 or a combination thereof.

99. The method of claim 97 or 98, wherein the subject has a chronic phase chronic myeloid leukemia (CML).

100. A method of preparing samples that are useful for stratifying a set of subjects having a chronic phase chronic myeloid leukemia (CML) for treatment, comprising:
a) obtaining or having obtained the samples from the subjects; and
b) reverse transcribing a miRNA from the individual samples to provide target oligodeoxynucleotides, wherein the miRNA is miR-15a, miR-15b, miR-16-1, miR-16-2 or a combination thereof.

101. The method of any one of claims 97-100, wherein the subject is a human.

102. The method of any one of claims 97-101, wherein the sample comprises bone marrow cells.

103. The method of any one of claims 97-101, wherein the sample comprises a blood sample.

104. The method of any one of claims 97-103, further comprising quantifying the target oligodeoxynucleotides in the sample prepared in step b).

105. The method of any one of claims 97-104, further comprising comparing the level of the target oligodeoxynucleotides in the sample with a threshold level.

106. The method of claim 105, wherein threshold level is the level of target oligodeoxynucleotides obtained from reverse transcription of a corresponding miR gene product in a control sample or a reference standard.

107. The method of claim 105 or 106, wherein:
a) the miR gene product is miR-15a, and the level of miR-15a in the sample is less than about 50% of the threshold miR-15a level;
b) the miR gene product is miR-15b, and the level of miR-15b in the sample is less than about 50% of the threshold miR-15b level;
c) the miR gene product is miR-16, and the level of miR-16 in the sample is less than about 50% of the threshold miR-16 level; or
d) a combination thereof.

108. The method of any one of claims 97-107, comprising reverse transcribing miR-15a, miR-15b, 16-1 and miR-16-2 in the sample to provide the target oligodeoxynucleotides.
